(19) United States
(12) Patent Application Publication
(10) Pub. No.: US 2011/0046887 A1
(43) Pub. Date: Feb. 24, 2011

(54) METHOD FOR ASSESSING PATHWAY PRODUCT LEVELS

(76) Inventors: Johannes D. Veldhuis, Rochester, MN (US); Daniel M. Keenan, Charlottesville, VA (US)

Correspondence Address: FISH & RICHARDSON P.C. (TC), PO BOX 1022, MINNEAPOLIS, MN, 55440-1022, US

(21) Appl. No.: 12/377,766

(22) PCT Filed: Aug. 15, 2007
(86) PCT No.: PCT/US07/76030

§ 371 Date: Oct. 6, 2010

Related U.S. Application Data
(60) Provisional application No. 60/822,610, filed on Aug. 16, 2006.

(51) Int. Cl. G01N 33/48

(52) U.S. Cl. 702/19

(57) ABSTRACT

This document relates to assessing homeostasis within mammals. For example, methods and materials for determining whether or not a biological system is functioning normally are provided.

### CROSS-REFERENCE TO RELATED APPLICATIONS

This application claims priority to U.S. Provisional Application No. 60/822,610, filed Aug. 16, 2006.

### STATEMENT AS TO FEDERALLY SPONSORED RESEARCH

This invention was made with government support under grant AG023133 awarded by the National Institute on Aging of the National Institute of Health and grant DK060717 awarded by the National Institute of Diabetes and Digestive and Kidney Diseases of the National Institute of Health. The government has certain rights in the invention.

### TECHNICAL FIELD

This document relates to healthcare, and more particularly to predicting disease.

### BACKGROUND

In the human, more than 26,500 gene products collaborate in a relatively unknown fashion to maintain health. A thesis of modern biology and medicine is that none of these signals acts alone. This axiom introduces the daunting challenge of parsing how interacting signals mediate homeostasis in the whole organism, organ, cell or organelle. Noninvasive methodologies are necessary to quantify regulatory pathways without disrupting underlying physiology.

### SUMMARY

This document discloses methods, systems, and computer program products for assessing pathway product levels in a mammal. In one aspect, this document discloses a method for assessing an inter-dependent biological system within a mammal. The method can include determining whether or not a set comprising at least three variables is within a diagnostic coordinate range. The set is obtained, using a mathematical formalism, from a measured value of a first constituent in a sample from the mammal and from a measured value of a second constituent in a sample from the mammal.

The first and second of the constituents can be constituents of the inter-dependent biological system, wherein the inter-dependent biological system can include at least three constituents. The diagnostic coordinate range can represent the inter-dependent biological system in normal function, wherein the presence of the set within the diagnostic coordinate range can indicate that the inter-dependent biological system of the mammal is normal. The absence of the set within the diagnostic coordinate range can indicate that the inter-dependent biological system of the mammal is abnormal.

In various implementations of the method, one or more of the following features may be included. The inter-dependent biological system can be a tripartite system. In one example, the inter-dependent biological system can include glucose, glucagon, and insulin. The first and second constituents can be selected from the group consisting of glucose, glucagon, and insulin. In some cases, the inter-dependent biological system can include testosterone, gonadotropin, and luteinizing hormone. In such cases, the first and second constituents can be selected from the group including testosterone, gonadotropin, and luteinizing hormone.

In other variations of the method, the diagnostic coordinate range can include control sets including at least three variables, wherein the sets are obtained, using the mathematical formalism, from a measured value of the first constituent from a collection of samples from healthy control mammals, and from a measured value of the second constituent in a collection of samples from healthy control mammals. In some variations of the method, the mammal can be a human. The range of variable values may include variable values sampled from a population of mammals, wherein the inter-dependent biological system is within the condition of homeostasis.

In yet other variations of the method, the mathematical formalism may include: a time delay signal for an agent to accumulate in a target tissue before it initiates a response; a nonlinear equation that represents concentration-dependent processes, and saturable cellular uptake and signaling processes; a stochastic term, to represent known measurement uncertainty and biological variability in dose-response properties; and signal parameter estimation procedures. The signal parameter estimation procedure can include maximum-likelihood and Bayesian approaches.

The time delay signal can be mathematically represented as a pulse, wherein the pulse is an administered dose of one of the three constituents. In some cases the administered dose can be a dose of glucose. In this case, the administered dose can be intravenously-administered glucose. The measured value of the first constituent can be measured from the sample as an activity or concentration level.

In another aspect, this document features a method for detecting the presence or absence of physiological regulatory failure in a mammal, as well as systems and computer program products that are used in executing the method. The method includes obtaining a biological sample, wherein the biological sample contains at least two constituents of an inter-dependent, tripartite biological system. The method further includes applying measured values of the two constituents to a mathematical formalism to obtain a set of deconvolved, time-independent variables within the biological system. The set of deconvolved time-independent variables can be compared to a diagnostic space coordinate exemplifying the tripartite biological system in normal function, wherein the coordinate position of the set of deconvolved, time-independent variables upon the diagnostic space coordinate can indicate the presence or absence of physiological regulatory failure in a mammal.

In other aspects, computer program products are provided that are tangibly embodied in an information carrier. The computer program products include instructions that, when executed, perform operations for executing the above-described methods and their variations. In yet further aspects, systems are provided that enable the above-described methods to be carried out.

Unless otherwise defined, all technical and scientific terms used herein have the same meaning as commonly understood by one of ordinary skill in the art to which this technology pertains. Although methods and materials similar or equivalent to those described herein can be used to practice those disclosed herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features, objects, and advantages will be apparent from the description and drawings, and from the claims.

### DESCRIPTION OF DRAWINGS

FIG. 1 is a diagram of a simplified tripartite control system.

FIG. 2 is a graph of a theoretical response surface.

FIG. 3 is a chart illustrating key sources of variability in biological feedback systems.

FIG. 4 is a diagram illustrating the fate of glucose within an exemplary biological system.

FIG. 5 is a diagram illustrating how a mathematical formalism can serve as the basis for an implementable model.

FIG. 6 is a graph plotting constant basal plus pulsatile saline or testosterone infusion.

FIG. 7 is a chart illustrating an algorithmic stratagem.

FIG. 8 is a graph plotting exponential regression of pulsatile luteinizing hormone output on measured concentrations of free testosterone.

FIG. 9 is a graph plotting regression of estimated inhibitory sensitivity or efficacy on age.

FIG. 10 is a graph plotting reconstruction of free testosterone's concentration-dependent negative feedback on luteinizing hormone secretion.

FIG. 11 is a chart representing (sample-discretized) luteinizing hormone secretion rates (left, top) driven by measured (injected) gonadotropin releasing hormone concentrations (left, bottom) with simultaneously estimated gonadotropin releasing hormone dose-response functions at each clamped stratum of testosterone availability (right).

FIG. 12 is a chart plotting analytical estimation of luteinizing hormone secretion, free testosterone concentrations and virtual gonadotropin releasing hormone drive.

FIG. 13 is a chart plotting joint response surface.

FIG. 14 is a simplified minimal schema.

FIG. 15 is a series of charts modeling estimates of insulin, glucose and glucagon secretion rates (in 4 pigs sampled frequently).

FIG. 16 is a series of charts showing model estimates in the same 4 pigs (as FIG. 15) after alloxan-induced diabetes in the animal.

FIG. 17 is a series of charts showing model estimates of insulin, glucose and glucagon concentrations (interrupted lines) in 4 separate pigs (intact, pre-alloxan).

FIG. 18 is a series of charts showing new estimates in the same 4 pigs (as FIG. 17) after diabetes was induced by alloxan.

FIG. 19 is a chart plotting glucose, glucagon, and insulin levels.

FIG. 20 is a chart plotting luteinizing hormone concentrations over time.

FIG. 21A is a series of charts showing the performance of a selective smoothing algorithm.

FIG. 21B is a series of charts showing the performance of a selective smoothing algorithm.

FIG. 22 illustrates an algorithmic flow.

FIG. 23 is a series of charts plotting hormone concentrations and secretory burst waveforms over time.

FIG. 24 is a series of charts plotting hormone concentrations and secretory burst waveforms over time.

FIG. 25 is a series of charts plotting Bayesian posterior mean and standard deviation of hormone levels versus time.

### DETAILED DESCRIPTION

Many techniques are available for assessing a level of disease in a patient, spanning both invasive and non-invasive methodologies. Examples of non-invasive modalities include imaging or fluid sampling (e.g., urine), whereas the more invasive techniques may involve tissue sampling and/or blood draws. Collected specimens are commonly analyzed for more than one component, where the level of the component can determine some degree of disease when compared with the same component concentration from a healthy population.

In some cases, measuring the levels of biological components can require both invasive and non-invasive techniques, such as comparing components of a blood draw with components of a tissue sample. However, it can be quite difficult to correlate the component levels from such a test if they are not taken at the same time and if the inter-dependence of the components cannot be substantially ignored. The ability to generate a “snapshot” of biological component concentrations at a given time, using a single technique, such as a single blood draw, may be very beneficial to those who diagnose disease.

Physiological homeostasis (i.e., a biological system in a state of physiological stability, or “normal” function) can require repeated incremental feedforward (agonistic) and feedback (inhibitory) adaptations within integrated systems. However, a major unmet need in clinical and laboratory research is an adequate noninvasive methodology to quantify in vivo signal exchange without disrupting biological linkages under study.

Intermittent feedforward can be illustrated in reproduction by gonadotropin-releasing hormone's (GnRH's) stimulation of luteinizing hormone (LH) synthesis in the pituitary gland; in puberty by growth hormone's induction of insulin-like growth factor type I gene expression in muscle; in metabolism by insulin's repression of liver glucose synthesis; and in mineral balance by parathyroid hormone's augmentation of bone anabolism.

Novel technologies are needed to quantify the strength of unobserved pathways that supervise multi-signal interactions noninvasively. Valid reconstruction of ensemble (network-like) control can markedly enhance the positive and negative predictive accuracies, sensitivity and specificity of disease identification (Pincus et al., Proc. Natl. Acad. Sci. USA 93:14100-14105 (1996); Keenan et al., Proc. Natl. Acad. Sci. USA 98:4028-403 (2001); Keenan et al., Proc. Natl. Acad. Sci. USA 101:6740-6745 (2004); Keenan et al., Endocrinol., Vol. 147(6), 2817-2828, (2006); Brandman et al., Science 310:496-498 (2005); Isaacs et al., Proc. Natl. Acad. Sci. USA 100:7714-7719 (2003); Klipp et al., Systems biology in practice, Weinheim, Germany. Wiley-VCH 3:449 (2005); Wolkenhauer, Biochem Soc Trans 33:507-515 (2005)). Thus, creation of a practicable ensemble construct can introduce a new echelon of investigative capabilities.

In several neurohormonal contexts, intermittent drive can be more effectual biologically than continuous stimulation. Pulsatile signal exchange can also mediate negative feedback, as illustrated by insulin and glucose's repression of glucagon secretion, cortisol's inhibition of adrenocorticotropin output, and sex-steroidal feedback on hypothalamic GnRH and pituitary luteinizing hormone (LH) outflow. A dearth of analytical tools to quantify episodic signal exchange in vivo is highlighted by review of 260,000 citations concerning dose-response analyses in the last decade (National Library of Medicine, August (2005)). There does not currently appear to exist a mathematically verified and experimentally validated methodology to quantify nonlinear concentration-dependent dose-response properties that mediate feedback by time-varying signals on observed and unobserved physiological pathways in the uninfused human or other mammal.

The term “pathways” is used herein to designate regulatory processes (dose-response interfaces or nonlinear transfer functions) that link biological inputs to cognate responses after a time delay. The following examples illustrate approaches to developing an accurate mathematical formalism of known physiological connections among an inter-dependent biological system.

### Glucose, Insulin, and Glucagon

A need for a paradigm shift in quantifying basic mechanisms of homeostatic glucose control is compelling, inasmuch as algorithmic strategies have not changed fundamentally in more than 2½ decades. To address existing model limitations and introduce a new investigative standard, this example describes a mathematical formalism, which can be used to: (1) develop a novel construct of interactive regulation among all three of glucose, insulin, and glucagon (and optionally somatostatin), thereby enhancing analytical power to detect subtle homeostatic failure; (2) incorporate a time delay for each signal to accumulate in its target tissue before it initiates a response, in accordance with known physiology (Chemick S S, Gardiner R J, Scow R O, Am J Physiol 253:E475-E480 (1987); Levy J R, Olefsky J M, Endocrinol 121:2075-2086 (1987); Yang Y J, Hope I D, Ader M, Bergman R N, J Clin Invest 84:1620-1628 (1989)); (3) implement nonlinear equations to correctly represent concentration-dependent and saturable cellular uptake and signaling processes (Keenan D M, Chattopadhyay S, Veldhuis J D, J Theor Biol; 236:242-255 (2005)); (4) allow for known measurement uncertainty and apparent biological variability in dose-response properties by including relevant stochastic terms (Keenan D M, Licinio J, Veldhuis J D, Proc Natl Acad Sci USA, 98:4028-4033 (2001); Keenan D M, Alexander S L, Irvine C H G, Clarke I J, Canny B J, Scott C J, Tilbrook A J, Turner A I, Veldhuis J D, Proc Natl Acad Sci USA, 101:6740-6745 (2004)); and (5) validate physiological structure in a dog model, and verify parameter estimability by direct mathematical proof. Stated technically, a desired construct (e.g., a mathematical formalism) can embody nonlinear dose-responsive coupling among all three of glucose, insulin and glucagon.

This example describes a method to detect and quantify the earliest stages of regulatory failure in nondiabetic individuals who may be at increased genetic or environmental risk for type II diabetes mellitus (DM) by analytically reconstructing pathways that mediate time-varying regulation of a minimal triad of interlinked signals, viz., glucose, insulin and glucagon. These methods can allow for the quantification of ensemble dynamics, rather than the behavior of any one signal, by estimating endogenous dose-response pathways noninvasively without using radiolabeled or otherwise marked molecules in awake unmedicated healthy individuals, given only sparse and noisy measurements of the three signals.

A technical objective of this example can include valid quantification of the potency and efficacy terms of the nonlinear dose-response functions interlinking glucose, insulin, and glucagon concentrations measured in the portal vein, hepatic vein, and peripheral artery of healthy unanesthetized dogs. A resultant analytical construct can be used in exploratory analyses of clinical data comprising glucose, insulin, and glucagon concentrations monitored repeatedly in peripheral blood after meal ingestion in both normoglycemic human patients, and patients with impaired fasting glucose (IFG) (vide infra). Three technical milestones of this method are to create, validate, and verify a construct that quantify tripartite interactions among glucose, glucagon, and insulin, in a non-invasive manner.

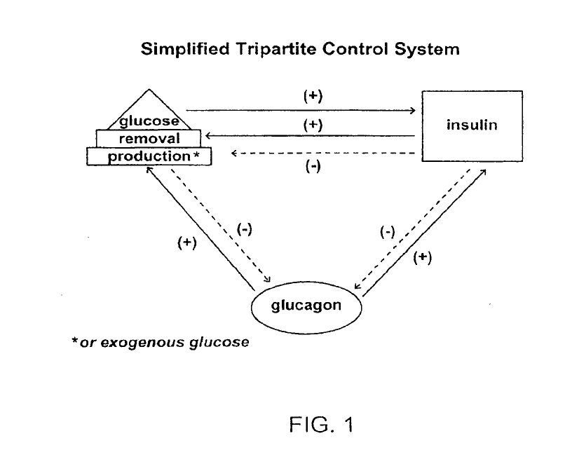

A mathematical charge is to frame a tractable analytical construct that can permit one to estimate the potency and efficacy of three-parameter logistic dose-response functions that interlink a triad of essential glucose-regulating signals. A mathematical formalism can be a nonlinear, mixed-effects model with time delays, in which deterministic and stochastic processes together transduce reciprocal feedback (repression) and feedforward (stimulation) by glucose, insulin, and glucagon (FIG. 1). Validation can involve simultaneous monitoring of all three signals in the dog portal vein every two minutes for one hour before and two hours after an intravenous (i.v.) glucose pulse. Intravenous rather than oral glucose administration or mixed-meal ingestion can be used initially in order to define a basic model before one incorporates somatostatin (SS) and other peptide signals, such as those mediating an incretin effect (Porksen et al., Diabetes, vol. 45:1317-1323 (1996); Silvestre et al., Eur J Pharmacol, vol. 469:195-200 (2003); Heller et al., Diabetes, vol. 46:785-791 (1997); Dunning et al., Diabetologia, vol. 48:1700-1713 (2005)).

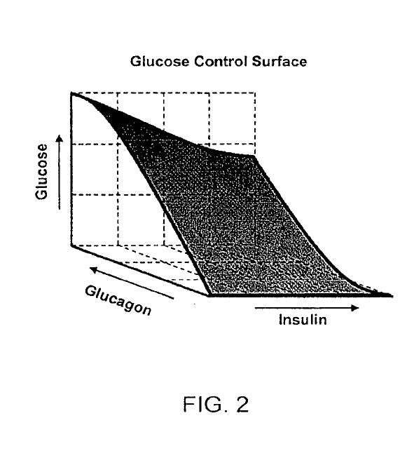

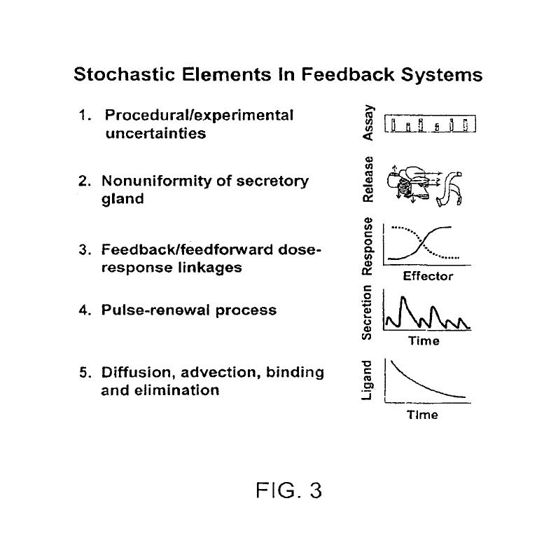

The essential physiological (deterministic) connections linking these three signals can be depicted by a three-dimensional response surface (FIGS. 1 and 2). A three-dimensional response surface such as that shown in FIG. 2 can be used as a diagnostic space coordinate. A diagnostic space coordinate can comprise the set of variables within a triad of signals that represent a healthy population, i.e., the range of variable sets for which the triad may be within the boundaries of normalcy, or homeostasis. A diagnostic space coordinate can describe boundaries for homeostatic conditions, to which a sample variable set (e.g., from a patient or a mammal to be evaluated) can be compared to diagnose a level of normal or abnormal homeostasis. Stochastic (apparently random) variations can be permitted to arise from combined sampling and assay errors (experimental uncertainty) and from efficacy estimates (system-parameter variability) (Keenan et al., Proc. Natl. Acad. Sci. USA, 101:6740-6745 (2004); Kennan et al., J Theor Biol., 236:242-255 (2005); Keenan et al., SIAM J. Appl. Math, 61:934-965 (2000)), FIG. 3.

Genetic risk of type II DM, aging, tumoral autonomy, and reduced negative feedback can elevate stochastic variability in diverse endocrine and metabolic systems, as quantified by the approximate entropy statistic (Pincus et al., Proc. Natl. Acad. Sci. USA 93:14100-14105 (1996); Schmitz et al., Am. J. Physiol. 35:E218-E226 (1997); Schmitz et al., Diabetes, 50:41-46 (2001); Meneilly et al., J. Clin. Endocrinol. Metab. 82:4088-4093, (1997); Meneilly et al., J. Clin. Endocrinol. Metab. 90:6251-6256 (2005); Pincus et al., Am. J. Physiol. 270:E107-E115 (1996); Pincus et al., Am. J. Physiol. 277:E948-E957 (1999); Veldhuis et al., Am. J. Physiol. 281:R1975-R1985 (2001); Keenan et al., Am. J. Physiol. 285:R664-R673 (2003); Hartman et al., J. Clin. Invest. 94:1277-1288 (1994); Pincus et al., Am. J. Physiol. 273:E989-E995 (1997)). To incorporate these fundamental properties, a mathematical model can comprise a set of coupled, time-delayed nonlinear differential equations with stochastic allowance, the general form of which can be as described elsewhere (Keenan et al., Proc. Natl. Acad. Sci. USA 98:4028-4033 (2001); Keenan et al., Proc. Natl. Acad. Sci. USA 101:6740-6745 (2004); Keenan et al., Endocrinol., Vol. 147(6), 2817-2828 (2006); Keenan et al., J. Theor. Biol. 236:242-255 (2005); Keenan et al., SIAM J. Appl. Math 61:934-965 (2000)).

A mathematical formalism, or, a core equation system, can embed physiological pathways linking glucose, insulin and glucagon via: (i) nominal time delays; (ii) nonlinear three-parameter logistic functions; (iii) secretion/appearance rates and disappearance kinetics; and (iv) projected local interstitial-fluid concentrations of each signal.

Regulatory interactions among glucose (Glc), insulin (Ins) and glucagon (Ggn), can be defined starting from first principles. For each of the three signals, feedback and feedforward regulatory strengths (potency, efficacy, sensitivity) as well as unobserved secretion rates from concentration-time profiles can be extracted. To this end, an overall circulatory system can be viewed topologically as a circle S¹ (an interval of length L with periodic boundary). Then, essential elements can be contained in the following construction. Let Cₖ^{(P)}(x,t) and Cₖ^{(T)}(x,t) denote plasma and tissue concentrations, and Z^{Endo}(x,t) and Z^{Exog}(x,t) the endogenous and exogenous rates of signal appearance at time 0≦t≦L and at location xεS¹, for k=Glc, Ins, Ggn. Let ηₖ⁽¹⁾ and ηₖ⁽²⁾ denote diffusion (mixing) and advection (linear-flow) coefficients along an axis of tubular capillary vessels; λₖ a difference in permeability of the endothelium to plasma and tissue fluids (Bassingthwaighte et al., The Cardiovascular System IV:549-626 (1984); Bassingthwaighte et al., Am. J. Physiol. 250:H539-H545 (1986)); and αₖ a rate of irreversible removal via metabolism or elimination. Then, plasma and tissue glucose and peptide dynamics can be described respectively as:

[Plasma]=−[Diffusion, Advection]−[Permeation into tissue]+[Delivery into Plasma]

$$\frac{\partial {C}_{k}^{( P )} ( x , t )}{\partial t} = {η}_{k}^{( 1 )} \frac{{\partial }^{2} {C}_{k}^{( P )} ( x , t )}{\partial {x}^{2}} - {η}_{k}^{2} \frac{\partial {C}_{k}^{( P )} ( x , t )}{\partial x} - {λ}_{k} ( {C}_{k}^{( P )} ( x , t ) - {C}_{k}^{( T )} ( x , t ) ) + ( {Z}_{k}^{Exog} ( x , t ) + {Z}_{k}^{Endo} ( x , t ) ) \qquad [ 1 ]$$

[Tissue]=[Permeation from Plasma]−[Degradation, Elimination]

$$\frac{\partial {C}_{k}^{( T )} ( x , t )}{\partial t} = {λ}_{k} ( {C}_{k}^{( P )} ( x , t ) - {C}_{k}^{( T )} ( x , t ) ) - {α}_{k} {C}_{k}^{( T )} ( x , t ) \\ k = Glc , Ins , Ggn \qquad [ 2 ]$$

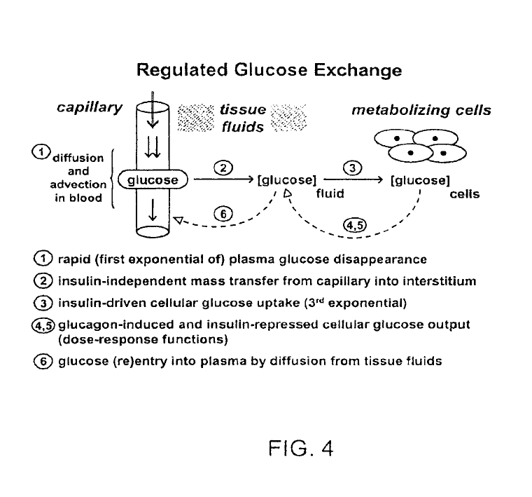

FIG. 4 illustrates the fate of glucose, which can be embodied by the above terms; viz. (1) rapid diffusion (random motion) and advection (linear distribution) in plasma; (2) delayed transfer across capillaries into interstitial fluid; (3) insulin-driven uptake into metabolically active cells; (4,5) insulin-repressed and glucagon-induced output of glucose from liver, muscle, and kidney into interstitial fluids; and (6) glucose entry from tissue fluid into plasma. Feedback and feedforward input signals (Fₖ^{(T)}(•)) can be assumed to be time-delayed, time-averages of the corresponding interstitial fluid concentrations, which can act via 4-parameter logistic functions. The interconnected secretion/appearance rates can be given for each of glucose, insulin, and glucagon, respectively, by:

$${Z}_{Glc}^{Endo} ( t ) = {β}_{Glc}^{( 0 )} + \frac{{β}_{Glc}^{( 4 )}}{1 + exp \{ - ( {β}_{Glc}^{( 1 )} + {β}_{Glc}^{( 2 )} \times {F}_{Ggn}^{( T )} ( t ) - {β}_{Glc}^{( 3 )} \times {F}_{Ins}^{( T )} ( t ) ) \}} \\ {Z}_{Ins}^{Endo} ( t ) = {β}_{Ins}^{( 0 )} + \frac{{β}_{Ins}^{( 4 )}}{1 + exp \{ - ( {β}_{Ins}^{( 1 )} + {β}_{Ins}^{( 2 )} \times {F}_{Glc}^{( T )} ( t ) - {β}_{Ins}^{( 3 )} \times {F}_{Ggn}^{( T )} ( t ) ) \}} \\ {Z}_{Ggn}^{Endo} ( t ) = {β}_{Ggn}^{( 0 )} + \frac{{β}_{Ggn}^{( 4 )}}{1 + exp \{ - ( {β}_{Ggn}^{( 1 )} + {β}_{Ggn}^{( 2 )} \times {F}_{Ins}^{( T )} ( t ) - {β}_{Ggn}^{( 3 )} \times {F}_{Glc}^{( T )} ( t ) ) \}} \qquad [ 3 ]$$

A physiological expectation can be that local (interstitial-fluid) insulin concentrations regulate glucose uptake from tissue fluids into liver, skeletal muscle and adipose cells nonlinearly. This relationship can be part of equation [2] and can be expressed via the 4-parameter function [4]:

$${α}_{Glc} = {γ}_{Glc}^{( 0 )} + \frac{{γ}_{Glc}^{( 3 )}}{1 + exp \{ {γ}_{Glc}^{( 1 )} + {γ}_{Glc}^{( 2 )} \times {F}_{Ins}^{( T )} ( t ) \}} \qquad [ 4 ]$$

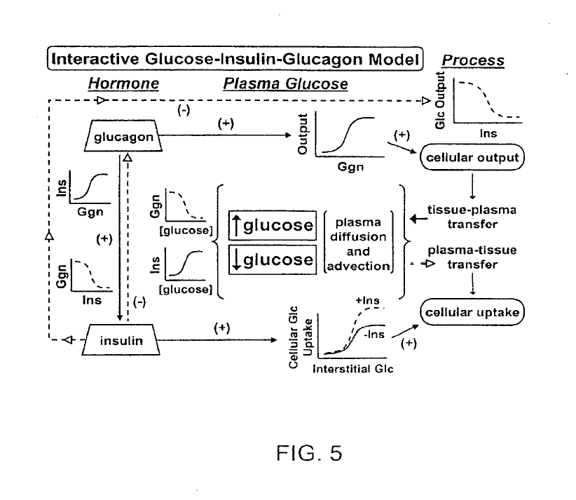

FIG. 5 shows how the above core equation system can serve as a basis for an implementable model. Because blood sampling is ordinarily performed at a single site x_{*}, it is oftentimes difficult to directly approximate spatial derivatives. One may need a representation that does not necessitate obtaining data at multiple spatial points (as required by the usual use of the associated Green's function). To this end, two complementary models can be used. In Model I, both plasma and tissue-fluid concentrations can be considered, with the unobserved tissue levels being reconstructed as part of the parameter estimation procedure. In Model II, plasma concentrations can be modeled, and dispersion and elimination can be viewed as being partitioned into compartments, three for glucose and two for each of insulin and glucagon.

Model I. Both the observed plasma, and unobserved tissue-fluid, concentrations can be modeled:

$$\begin{matrix}\frac{\partial {C}_{k}^{( P )} ( t )}{\partial t} = - {η}_{k} {C}_{k}^{( P )} ( t ) - {λ}_{k} ( {C}_{k}^{( P )} ( t ) - {C}_{k}^{( T )} ( t ) ) + {Z}_{k} ( t ) & [ 5 ] \\ \frac{\partial {C}_{k}^{( T )} ( t )}{\partial t} = {λ}_{k} ( {C}_{k}^{( P )} ( t ) - {C}_{k}^{( T )} ( t ) ) - {α}_{k} {C}_{k}^{( T )} ( t ) & [ 6 ] \\ k = Glc , Ins , Ggn & \end{matrix}$$

Model II. The following convolutions can be valid approximations of Equations [1]-[2] for plasma concentrations:

$$\begin{matrix}{C}_{Glc}^{( P )} ( t ) = {C}_{Glc}^{( P )} ( 0 ) ( \begin{matrix}{a}_{Glc}^{( 1 )} {e}^{- {α}_{Glc}^{( 1 )} t} + \\ {a}_{Glc}^{( 2 )} {e}^{- {α}_{Glc}^{( 2 )} t} + \\ {a}_{Glc}^{( 3 )} {e}^{- {α}_{Glc}^{( 3 )} t}\end{matrix} ) + {\int }_{0}^{t} ( \begin{matrix}{a}_{Glc}^{( 1 )} {e}^{- {α}_{Glc}^{( 1 )} ( t - r )} + \\ {a}_{Glc}^{( 2 )} {e}^{- {α}_{Glc}^{( 2 )} ( t - r )} + \\ {a}_{Glc}^{( 3 )} {e}^{- {α}_{Glc}^{( 3 )} ( t - r )}\end{matrix} ) {Z}_{Glc} ( r ) d r \\ {C}_{Ins}^{( P )} ( t ) = {C}_{Ins}^{( P )} ( 0 ) ( \begin{matrix}{a}_{Ins}^{( 1 )} {e}^{- {α}_{Ins}^{( 1 )} t} + \\ {a}_{Ins}^{( 2 )} {e}^{- {α}_{Ins}^{( 2 )} t}\end{matrix} ) + {\int }_{0}^{t} ( \begin{matrix}{a}_{Ins}^{( 1 )} {e}^{- {α}_{Ins}^{( 1 )} ( t - r )} + \\ {a}_{Ins}^{( 2 )} {e}^{- {α}_{Ins}^{( 2 )} ( t - r )}\end{matrix} ) {Z}_{Ins} ( r ) d r \\ {C}_{Ggn}^{( P )} ( t ) = {C}_{Ggn}^{( P )} ( 0 ) ( \begin{matrix}{a}_{Ggn}^{( 1 )} {e}^{- {α}_{Ggn}^{( 1 )} t} + \\ {a}_{Ggn}^{( 2 )} {e}^{- {α}_{Ggn}^{( 2 )} t}\end{matrix} ) + {\int }_{0}^{t} ( \begin{matrix}{a}_{Ggn}^{( 1 )} {e}^{- {α}_{Ggn}^{( 1 )} ( t - r )} + \\ {a}_{Ggn}^{( 2 )} {e}^{- {α}_{Ggn}^{( 2 )} ( t - r )}\end{matrix} ) {Z}_{Ins} ( r ) d r\end{matrix} \qquad [ 7 ]$$

What one may observe experimentally is a discrete-time sampling:

Y_{Glc,i}=C_{Glc}^{(P)}(tᵢ)+ε_{Glc,i}, Y_{Ins,i}=C_{Ins}^{(P)}(tᵢ)+ε_{Ins,i}, Y_{Ggn,i}=C_{Ggn}^{(P)}(tᵢ)+ε_{Ggn,i}, 1=1, . . . , n,

where the ε's can be IID Gaussian. Because of nonlinear interactions introduced by the secretion rates, the likelihood function (L) may not be Gaussian. Thus, for cross-validation, estimation approaches can be utilized, such as: (i) Likelihood-Based and (ii) Bayesian.

I. Likelihood-Based Estimation Procedure.

The maximum-likelihood estimation (MLE) approach can entail the following four steps:

Step 1. Using Model II, the observed plasma Glc, Ins and Ggn concentrations and population-specific rates of elimination (α⁽¹⁾, α⁽²⁾, α⁽³⁾), the time-varying secretion (or appearance) rates: \hat{Z}_{Glc}(•), \hat{Z}_{Ins}(•), and \hat{Z}_{Ggn}(•) can be estimated via regularization constraints, for k=Glc, Ins, Ggn, (m=2 or 4), with smoothing parameter κ:

Minimize_{Z} {−log L(Zₖ|Data)+κ∥D^{m}Z∥²} [8]

That is, secretion rates can be first estimated independently of feedback and feedforward modulating signals. Since any administered glucose, insulin or glucagon can be intravenous, exogenous rates can be known and removed from the estimate of total secretion rates, obtaining: \hat{Z}_{Glc}^{Endo}(•), \hat{Z}_{Ins}^{Endo}(•) and \hat{Z}_{Ggn}^{ENdo}(•).

Step 2. Using equation [4], Step 1, estimated secretion rates \hat{Z}_{Glc}(•), \hat{Z}_{Ins}(•) and \hat{Z}_{Ggn}(•), one can estimate tissue-fluid concentrations: Ĉ_{Glc}^{(T)}(•), Ĉ_{Ins}^{(T)}(•) and Ĉ_{Ggn}^{(T)}(•) for any choice of the parameters in equations [5]-[6]. Hence, one can write down a likelihood function and obtain MLE estimates of the parameters and the corresponding tissue concentrations. Using these, and population-specific time delay values for feedback and feedforward, one can achieve estimates of the feedback and feedforward signals: \hat{F}_{Glc}^{(T)}(•), \hat{F}_{Ins}^{(T)}(•) and \hat{F}_{Ggn}^{(T)}(•).

Step 3. Using \hat{Z}_{Glc}^{Endo}(•), \hat{Z}_{Ins}^{Endo}(•) and \hat{Z}_{Ggn}^{Endo}(•), and \hat{F}_{Glc}^{(T)}(•), \hat{F}_{Ins}^{(T)}(•) and \hat{F}_{Ggn}^{(T)}(•), the parameters for secretion and insulin-dependent glucose uptake can be estimated in the logistic equations [3]-[4].

Step 4. One can return to Step 1, and now estimate all or some of the previously assumed fixed rates of elimination.

II. Bayesian Estimation Procedure. Suppose that the parameters in equations [3 ]-[7] are represented as θ=(θ₁, θ₂, . . . , θᵣ). The Bayesian approach can create a posterior probability density of the parameters: p(θ|Data), utilizing the likelihood (L(θ|Data)) and prior information via a prior probability density p(θ): p(θ|Data)∝L(θ|Data) p(θ).

The Gibbs' method (Grenander et al., Brown University, Providence, R.I. (1983); Geman et al., IEEE Trans Pattern Anal Machine Intell 6:721-741 (1984)) can allow one to simulate from the posterior distribution multiple times and thus accurately estimate any desired posterior probability for the parameters. The Bayesian method has become one of the most applied statistical methods (Gelfand A, Smith A F M, Journal of American Statistical Association 85:398-409 (1990)). To apply the method, one may need to explicitly calculate the posterior density for each component of the parameter, conditioned on all the other parameters and the data.

This computation is often difficult to accomplish, especially when there are nonlinear interactions, such as in the equations [3]-[8], above. Grenander et al. (1990) introduced a method, Factored Sampling, to circumvent this difficulty (Grenander et al., HANDS: A Pattern Theoretic Study of Biological Shapes. Springer-Verlag, New York (1990)). One then may only need to simulate from the prior density (and to evaluate the likelihood function). In the present case, Factored Sampling can allow one to obtain posterior probability distributions for the parameters in equations [3]-[8].

Factored Sampling. Suppose that the parameter set is θ=(θ₁, θ₂, . . . , θᵣ), and that a likelihood function L(θ|Data), and a prior probability density p(θ), θεΘ, are given. An initial value of θ can be chosen, θ=θ⁽⁰⁾. For the 1^{st} component of θ, one can generate n values from the prior density for θ₁, call them: θ_{(1,1)}, θ_{(1,2)}, . . . , θ_{(1,n)}. One can evaluate each of these in the likelihood: L((θ_{(1,j)}, θ₂⁽⁰⁾, θ₃⁽⁰⁾, . . . , θᵣ⁽⁰⁾)|Data), j=1, . . . , r, and convert the r likelihood values to probabilities by dividing each by the total of all r likelihood values. With these probabilities, one can then randomly select one of the θ_{(1,1)}, θ_{(1,2)}, . . . , θ_{(1,n)}; call it θ_{(1,j)}. This value can be substituted into the 1st component of θ, and the same procedure can be repeated in the 2nd component, 3rd, . . . , r-th component. One can repeat the process (updating each of the r components) a large number of times. The result (asymptotically) can be an estimated value \hat{θ}=(\hat{θ}₁, \hat{θ}₂, . . . , \hat{θ}ᵣ), which is a simulation from the posterior density for θ. This is one simulation, which then can be repeated a large number of times, N.

The resulting simulations can allow one to calculate any desired posterior probability. For example, the posterior probability of a r-dim set A can be the relative number (approximately) of the N simulations that fall in set A. Given the foregoing methodology the particular steps of the Bayesian approach can be as follows.

Step 1. The secretion/appearance rates: \hat{Z}_{Glc}(•), \hat{Z}_{Ins}(•), and \hat{Z}_{Ggn}(•), can be obtained as described in the Likelihood-Based approach. The regularization method, Minitnize_{Z} {−log L(Zₖ|Data)+κ∥D^{m}Z∥²}, where (m=2 or 4), can be interpretable as a Bayesian method that can produce the secretion rate Z, which is the posterior mode under a prior probability distribution (on Z's) that is proportional to e^{-κ∥D}^{^{m}}^{Z∥}^{²}.

Step 2. Using Factored sampling, we can obtain posterior distributions for the parameters in equation [5]-[6], and posterior estimates of tissue concentrations, Ĉ_{Glc}^{(T)}(•), Ĉ_{Ins}^{(T)}(•), and the feedback and feedforward signals: \hat{D}_{Glc}^{(T)}(•), \hat{F}_{Ins}^{(T)}(•) and \hat{F}_{Ggn}^{(T)}(•).

Step 3. Using Factored Sampling, we can then obtain posterior distributions for the logistic parameters of secretion and insulin-dependent glucose uptake in equation [3]-[4].

An approach can involve the following parameters:
(i) time delays for glucose, insulin and glucagon interactions can be included explicitly by way of the intervals required for the three signals to undergo diffusion and advection in plasma and leave plasma and reach their local sites of action in interstitial fluid (Yang et al., J. Clin. Invest. 84:1620-1628 (1989); Bodenlenz et al., Am. J. Physiol. Endocrinol. Metab. 289:E296-E300 (2005); Nielsen et al., Diabetes 54:1635-1639 (2005); Abi-Saab et al., J. Cereb. Blood Flow Metab. 22:271-279 (2002); Schwartz et al., Am. J. Physiol. 259:t-83 (1990); Butler et al., Am. J. Physiol. 264:E548-E560 (1993); Steil et al., Am. J. Physiol. 271:E855-E864 (1996); Hamilton-Wessler et al., Diabetes 51:574-582 (2002)), FIG. 4;
(ii) input signals to the dose-response functions can be defined by their projected interstitial fluid (rather than measured plasma) concentrations (Ito et al., Metabolism 44:358-362 (1995); Poulin et al., Diabetes 43:180-190 (1994); Gupta et al., Am. J. Physiol. Endocrinol. Metab. 283:E1002-E1007 (2002); Raju et al., Diabetes 54:757-764 (2005); Haaparanta et al., Life Sci. 73:1437-1451 (2003); Cline et al., New England Journal of Medicine 341:240-246 (1999); Muller et al., Am. J. Physiol. 271:E1003-E1007 (1996)). Thus, dose-response functions can be time-delayed with respect to plasma for both inputs and responses (Raskin et al., J. Clin. Invest. 56:1132-1138 (1975); Edgerton et al., J. Clin. Invest. 116:521-527 (2006); Edgerton et al., Diabetes 50:1872-1882 (2001); Basu et al., Diabetes 53:2042-2050 (2004); Barzilai et al., J. Biol. Chem. 268:25019-25025, (1993); Rizza et al., Am. J. Physiol. 240:E630-E639 (1981); Byrne et al., Am. J. Physiol. 268:E21-E27 (1995)), FIG. 5;
(iii) glucose uptake from interstitial fluid into target cells can be driven by a nonlinear insulin concentration-response function (Haaparanta et al., Life Sci. 73:1437-1451 (2003); Cline et al., Am. J. Physiol. 274:E381-E389 (1998); Thome-Duret et al., Anal Chem 68:3822-3826 (1996); Regittnig et al., Am. J. Physiol. Endocrinol. Metab. 285:E241-E251, (2003));
(iv) initial exocytotic peptide release (first-phase insulin secretion) can be assumed to occur rapidly given that (a) i.v. glucose can induce maximal insulin concentrations in the portal vein within 1 minute and in systemic blood within two-five minutes and (b) the duration of individual insulin and glucagon secretory bursts can be one-two minutes in the human, dog, monkey, pig and rat (Porksen et al., Am. J. Physiol. 269:E1106-E1114 (1995); Goodner et al., Science 195:177-179 (1977); Stagner et al., J. Clin. Invest. 65:939-942 (1980); Ito et al., Metabolism 44:358-362 (1995); Porksen et al., Am. J. Physiol. 282:E695-E702 (2002); Song et al., J. Clin. Endocrinol. Metab. 82:4491-4499 (2000); Ritzel et al., J. Clin. Endocrinol. Metab. 88:742-747 (2003); Ritzel et al., Am. J. Physiol. Endocrinol. Metab. 290:E750-E756 (2006); Porksen et al., Diabetes 51:S245-S254 (2002); Laedtke et al., Am. J. Physiol. 279:E520-E528 (2000); Paolisso et al., J. Clin. Endocrinol. Metab. 66:1220-1226 (1988); Mitrako et al., New England Journal of Medicine 326:22-29 (1992); Alberti et al., Lancet 2:1299-1301 (1973)); and
(v) delayed peptide release (slow-phase insulin secretion) can be assumed to occur gradually after a time delay, possibly via the slower dissolution of (nonfluid-phase) insulin-zinc crystals within secretory granules.

The projected outcome can be a reconstructed three-dimensional response surface,

FIG. 2, in which the plasma glucose concentration can be jointly determined not only by its biexponential disappearance kinetics, but more expressly by time-advanced insulin, glucagon and glucose concentrations acting through cognate dose-response functions (FIG. 5). In the MLE model, a precision of potency and efficacy estimates can be evaluated via bootstrap resampling; i.e. by refitting an entire response surface 1000 times after randomly reassigning the residuals (Keenan et al., Endocrinol., Vol 147(6), 2817-1818, (2006)). In the case of the Bayesian model, parameter precision can be assessed by sampling repeatedly from a posterior probability distribution (Keenan et al., J. Theor. Biol. 236:242-255 (2005)). In an analysis of dose-response properties in the GnRH, LH and testosterone ensemble (EXAMPLE II), the CV (SD/mean×100%) of MLE parameter estimates was 3-15% and of Bayesian estimates 8-21% (Keenan et al., Endocrinol. Vol 147(6), 2817-1818, (2006); Keenan et al., J. Theor. Biol. 236:242-255 (2005)). The latter depend in part on the assumed precision of the priors, as well as model fit and the data.

Primary experimental validation can be accomplished in an animal model. Five core signals, viz. glucose, insulin, glucagon, SS and C-peptide, can be quantified concurrently by direct catheterization of the portal vein, a hepatic vein and a peripheral artery in conscious dogs. To validate model formulation, blood can be sampled every two min for three hours—for 1 hour fasting and for two hours after bolus i.v. infusion of a 7 gm glucose pulse (Porksen et al., Am. J. Physiol. 269:E1106-E1114 (1995); Porksen et al., Diabetes 45:1792-1797 (1996); Porksen et al., Diabetes 45:1317-1323 (1996); Porksen et al., Am. J. Physiol. 270:E1043-E1049 (1996); Porksen et al., Am. J. Physiol. 269:E478-E488 (1995)). IV glucose can be used here to obviate confounding by corollary effects of gut-related (incretin) peptides. A clinical interventional radiological approach of percutaneous transhepatic portal-venous sampling can be adopted instead of chronic multivessel catheterization. Frequent sampling may be needed for primary model validation, inasmuch as second-exponential half-lives of disappearance of plasma insulinotropic peptides can be 2.9-6.6 min and ratios of their slow/total decay amplitudes can be 0.18-0.39 (Porksen et al., Am. J. Physiol. 269:E1106-E1114 (1995); Porksen et al., Am. J. Physiol. 282:E695-E702 (2002); Song et al., J. Clin. Endocrinol. Metab. 82:4491-4499 (2000); Laedtke et al., Am. J. Physiol. 279:E520-E528 (2000); Ho et al., Clin. Physiol. Biochem. 4:257-267 (1986); Alford et al., J. Clin. Endocrinol. Metab. 42:830-838 (1976)). Validation can require complete data collection in five animals (90 measurements of glucose and each of the four peptides in each dog).

A point of sampling at pre- and posthepatic locations simultaneously is to exploit signal/noise ratios of 6-8 in the portal vein and to compute the fractional extraction of peptides across the liver before their dissipation within the systemic circulation (Porksen et al., Am. J. Physiol. 269:E1106-E1114 (1995); Porksen et al., Am. J. Physiol. 282:E695-E702 (2002); Porksen et al., Am. J. Physiol. 269:E478-E488 (1995); Song et al., J. Clin. Endocrinol. Metab. 82:4491-4499 (2000); Alford et al., Clin Endocrinol. (Oxf) 11:413-424 (1979); Sindelar et al., Diabetes 45:1594-1604 (1996)). In the case of peripherally measured insulin, portal-vein insulin secretion can be estimated analytically using concomitant peripheral C-peptide concentrations, given negligible hepatic removal of C-peptide and equimolar release of the two peptides (Toffolo et al., Am. J. Physiol. Endocrinol. Metab. 290:E169-E176 (2006)). For glucagon and somatostatin, a concentration-dependent hepatic extraction can be calculated directly in each animal (based on 90 paired pre- and posthepatic measurements).

A longer t_{1/2} of systemic C-peptide can damp its fractional pulse amplitude compared with that of insulin, thus partially censoring pulse enumeration. Accordingly, deconvolution analyses can be modified to exploit the combined information inherent in paired C-peptide and insulin molar concentration time series, as suggested by others (Eaton et al., J. Clin. Endocrinol. Metab. 51:520-528 (1980); Polonsky et al., J. Clin. Invest. 72:1114-1123 (1983); Van Cauter et al., Diabetes 41:368-377 (1992)).

Plasma glucose concentrations can be measured in triplicate by a glucose oxidase technique [Glucose Analyzer 2, Beckman Coulter Inc., Palo Alto, Calif.] immediately upon sampling in an animal-procedures suite. The 4 peptides can be quantified using trasylol-preserved (1000 IU/mL) thawed plasma. The assays can be ELISA and solid-phase double-antibody assay as follows: insulin [Novo Nordisk, Bagsvaerd, DK], C-peptide [DAKO Ltd., Cambridgeshire, UK], and glucagon and SS14 [Alpco Diagnostics, Salem, N.H.], validated for recovery and parallelism using human standards (Stagner et al., Diabetes 37:1715-1721 (1988); Porksen et al., Am. J. Physiol. 278:E162-E170 (2000); Song et al., Endocrinol. 144:3399-3405 (2003); Ritzel et al., J. Clin. Endocrinol. Metab. 88:742-747 (2003); Ritzel et al., Am. J. Physiol. Endocrinol. Metab. 290:E750-E756 (2006); Porksen et al., Diabetes 51:S245-S254 (2002); Schmitz et al., Diabetes 50:41-46 (2001); Shah et al., Am. J. Physiol. 277:E283-E290 (1999); Toffolo, et al., Am. J. Physiol. Endocrinol. Metab. 290:E169-E176 (2006)).

MLE and Bayesian estimates of key dose-response parameters are not expected to differ significantly. Differences arise with respect to absolute confidence intervals (see above) and relative computational burden (Keenan et al., Endocrinol. Vol 147(6), 2817-1818, (2006); Keenan et al., J. Theor. Biol. 236:242-255 (2005)). Implementing both statistical approaches can be important for cross-validation of the new model.

Optimizing a sampling schedule and minimizing a core parameter set post hoc can allow one to frame the most frugal protocol for later clinical use. An irregularly spaced sampling schema can be necessary, given the distinct kinetics of glucose, insulin and glucagon action, response, appearance, and disappearance. A point is to establish such estimates by framing and validating the primary model comprehensively in the dog.

To explore robustness of a three-signal construct validated by two-min sampling in the portal-vein of the dog, the constituent 4, 8, 12, 20 and 30-min data subsets can be separately analyzed. An objective is to delineate the impact of data density on parameter precision, defined by a coefficient of variation. Analogous sensitivity analyses can be performed to define optimally irregular spacing of samples for an idealized clinical procedure. A goal can be nominal parameter precision of 5-15% for dose-response potency and efficacy estimates. A next challenge involves minimizing sampling requirements and parameters to facilitate clinical application. For example, solving for 15 parameters from three signals each measured 20 times could leave approximately 45 degrees of freedom. The method may: (1) have immediate utility in cross-sectional and prospective clinical studies; (2) establish a framework for adding other insulinotropic signals to the model (Table 1); and (3) establish proof-of-concept for more general application to other metabolic networks.

One exploratory question may be the merit of later adding SS to create a 4-signal model of glucose control. This question can be explored using SS data obtained in the dog. A second exploratory query may be the practicability of analyzing 13 consecutive measurements of glucose, insulin, C-peptide and glucagon in normal subjects and in patients with IFG after ingestion of a mixed meal.

TABLE 1
Ensemble Concept of Homeostasis

Insulin
1. ↑ glucose disappearance
2. ↓ glucose production
3. ↓ glucagon secretion
Glucagon
1. ↑ glucose production
2. ↑ insulin secretion
Glucose
1. ↑ insulin secretion
2. ↓ glucagon secretion

A third issue can be the possibility of adapting a basic three-signal model to allow group analyses of data in cohorts of subjects with normoglycemia, IFG or impaired glucose tolerance. A concept is to formulate cohort-defined parameter estimates and statistical confidence intervals, thus permitting comparisons among groups. An advantage of this strategy can be that estimating any given parameter for the cohort as a whole yields a marked increase in the number of degrees of freedom, since data from all subjects can be used concurrently. A cohort-parameterization concept is illustrated below in estimating the potency and efficacy of hypothalamic GnRH's virtual drive of LH secretion, testosterone's feedback on LH, and LH's stimulation of testosterone output (25 parameters) noninvasively in separate groups of 10 young and 8 older men.

Whereas experimental data obtained in the awake dog can ensure primary empirical validation of physiological assumptions, direct statistical verification of parameter identifiability may also be obtained. By way of analogy, three PhD theses in statistics and two technical and two applied-mathematics manuscripts have verified overall system realizability and parameter asymptotics for a three-signal neuroendocrine ensemble, also defined by nonlinear, time-delayed deterministic and stochastic dose-response linkages (Keenan et al., Proc. Natl. Acad. Sci. USA 98:4028-4033 (2001); Keenan et al., Proc. Natl. Acad. Sci. USA 101:6740-6745 (2004); Keenan et al., J. Theor. Biol. 236:242-255 (2005); Keenan et al., SIAM J. Appl. Math 61:934-965 (2000)).

The algorithms can be developed in software programming packages. An exemplary software programming package is MATLAB®. The parameter-estimation procedures described herein may be found in the MATLAB® Optimization suite and the complementary Genetic and Direct-Search algorithms. The latter methods can be important for optimization problems, in which an objective function is discontinuous, highly nonlinear, stochastic or susceptible to unreliable or undefined derivatives. While traditional optimization algorithms depend upon information about the gradient or higher derivatives, the genetic and direct-search methods repeatedly modify a population of individual minima using rules modeled on gene recombination. This can be necessary to achieve a true global minimum. Because genetic algorithms may not be so tractable for nonlinear estimation problems, two direct-search methods can be suitable: generalized pattern search (GPS) and mesh-adaptive search (MADS). Both techniques can be based upon delineating minimal and maximal positive basis patterns. MADS can be appropriate for the present focus.

After validation, an ensemble model can be made available in various formats. For example, MATLAB® code (text files) that can be run using MATLAB® on any compatible platform; and binary executable code for Windows®, Macintosh®, Linux, and Solaris platforms, that do not require the use of MATLAB®. An interactive tool GUIDE (Graphical User Interface Development Environment) can allow for the creation of a user-friendly graphical interface including list boxes, pull-down menus, push buttons, radio buttons, sliders and plots. An outcome can be a self-contained application for end-users.

The following example illustrates an approach to developing an accurate mathematical representation of known physiological connections among gonadotropin-releasing hormone (GnRH), luteinizing hormone (LH) and testosterone (Te); primary empirical validation of an interconnected structure in an animal model; and direct statistical verification of parameter identifiability.

### Gonadotropin-Releasing Hormone (GnRH), Luteinizing Hormone (LH), and Testosterone (Te)

This example focuses on noninvasive reconstruction of signal exchange, taking as an illustrative clinical problem an unexplained age-related decline in testosterone (Te) availability in healthy men. Te deficiency in adults can predict osteopenia, sarcopenia, cardiovascular risk, visceral adiposity, insulin resistance, hypertension, decreased aerobic capacity, cognitive impairment and reduced quality of life. A technical challenge arises, inasmuch as: (a) androgen availability can be governed by time-delayed interactions among all three of hypothalamic GnRH, pituitary LH and testicular Te, rather than by any single effector considered in isolation; (b) Te secretion can inhibit both measurable LH pulses and unobserved GnRH signals; (c) negative feedback by Te can putatively operate via time-delayed, nonlinear concentration-dependent functions with jointly deterministic and stochastic properties; (d) an unknown fraction of plasma (protein-bound or unbound) Te can suppress the hypothalamus and pituitary gland; (e) each signal (GnRH, LH and Te) can be pulsatile, yet data obtained in vivo can be often sparse and noisy; and (f) reproductive system output should be monitored informatively without disrupting ensemble control.

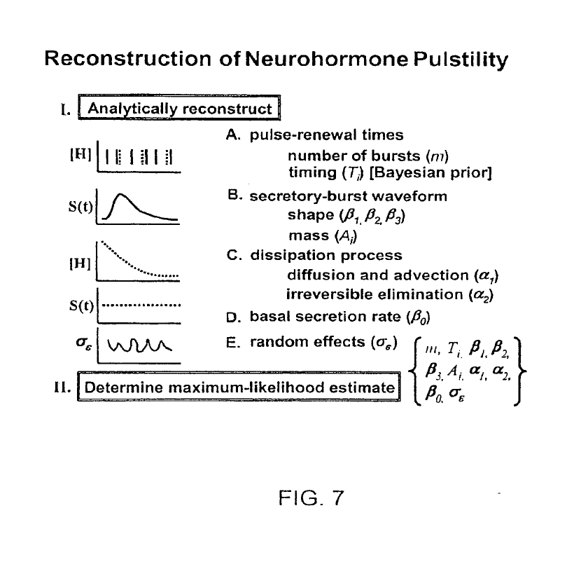

Volunteers (Forty (40) healthy men ages 18-80 years) arrived at a testing facility by 1730 hours and stayed two nights. At 1800-2000 hours, two i.v. catheters were placed in (contralateral) antecubital veins to allow concomitant saline or Te infusion and blood sampling. At midnight (MN), a steroidogenically inhibitory dose of ketoconazole (KTCZ 1000 mg) and a replacement amount of glucocorticoid (dexamethasone, DEX 0.75 mg) was given orally (Veldhuis et al., Am. J. Physiol. 272:R464-R474 (1997); Zwart et al., J. Clin. Endocrinol. Metab. 82:2062-2069 (1997)). This regimen can reduce the concentration of total Te from 530±61 to 85±13 ng/dL overnight. Additional doses of KTCZ (400 mg) were administered the next day at 0600 hours, 1200 hours and 1800 hours (drug half-life ˜six hours) and a second dose of DEX (0.75 mg) at 1200 hours (since KTCZ induces DEX metabolism) (FIG. 7). For safety reasons, DEX (0.75 mg, half-life ˜22 hours) were administered orally at the end of each GCRC admission. Visits were greater than or equal to two weeks apart. The order of the four visits was prospectively randomized, and interventions were double-blind.

To fix basal Te availability, a constant 24-hours i.v. infusion of Te was started at MN right after the loading dose of KTCZ/DEX. To enforce pulsatile negative feedback, 12 consecutive pulses of Te or saline was given i.v. on top of the basal infusion. To assess any changes in GnRH potency or efficacy, GnRH was injected by i.v. bolus at 2000 hours (33 ng/kg) and at 2200 hours (1.5 μg/kg). To monitor LH and Te concentrations, blood samples (1.25 mL) were withdrawn every 10 min for 16 hours beginning at 0800 hours, FIG. 7. The 8-hours delay between starting Te infusion and blood sampling encompasses greater than 10-fold the t_{1/2} of total Te and greater than 100-fold that of free Te (Veldhuis et al., Physiological Basis of Aging and Geriatrics, 3rd edition, 213-231 Boca Raton, CRC Press (2003); Veldhuis, Reproductive Endocrinology 4th edition, 622-631, Philadelphia, W.B. Saunders Co., (1999). Blood was withdrawn additionally at −30, −20, −10, 0 (just before the GnRH bolus), 1, 2.5, 5, 7.5, 12.5, 15, 20, 30, 40 and 60 minutes to measure injected GnRH concentrations, given that endogenous GnRH is undetectable (Veldhuis et al., Physiological Basis of Aging and Geriatrics, 3rd edition, 213-231, Boca Raton, CRC Press (2003); Veldhuis, Reproductive Endocrinology, 4th edition, 622-631 Philadelphia, W.B. Saunders Co., (1999)).

Te was infused at a constant rate of 1.0 mg/24 hours from midnight (MN) until MN. There were 12 superimposed pulses each at a session-defined dose of zero (saline), 167, 333 or 500 μg delivered over 6 min every two hours from MN to 2200 hours. Resultant total (basal+pulsatile) Te infusion rates were equivalent to 1, 3, 5 and 7 mg/24 hours, thus spanning sub-, mid- and high-physiological Te production rates in healthy young men (normal interquartile range 4-6 mg/day) (Veldhuis et al., Physiological Basis of Aging and Geriatrics, 3rd edition, 213-231, Boca Raton, CRC Press (2003); Veldhuis, Reproductive Endocrinology, 4th edition, 622-631, Philadelphia, W.B. Saunders Co., (1999)). Crystalline testosterone was dissolved in 100% ethanol, diluted to less than 0.3% ethanol in normal saline, and microfiltered for i.v. infusion after sterility and pyrogen testing.

LH and total Te concentrations were determined in duplicate in each serum sample via ultrasensitive, double-monoclonal, robotics-automated chemiluminescence assays (Veldhuis et al., J. Clin. Endocrinol. Metab. 84:3498-3505 (1999); Veldhuis et al., J. Clin. Endocrinol. Metab. 85:1477-1486 (2000)). LH and Te concentrations in this platform were uniformly detectable (>3 SD's above hypopituitary serum) in prepubertal children and older adults (Mulligan et al., J. Clin. Endocrinol. Metab. 86:5547-5553 (2001); McCartney et al., Am. J. Physiol. Endocrinol. Metab. 286:E902-E908 (2004). Pooled (30-min) samples were used to assay free Te (by equilibrium dialysis at 37 C), nonSHBG-bound [bioavailable] Te (after 50% (NH₄)₂SO₄ precipitation), albumin and SHBG concentrations (Mulligan et al., Eur. J. Endocrinol. 141:257-266 (1999); Veldhuis et al., J. Clin. Endocrinol. Metab. 84:3506-3514 (1999); Potischman et al., Cancer Res. 54:5363-5367 (1994); Bhasin et al., Am. J. Physiol. 281:E1172-E1181 (2001)). The Te assay correlates closely with GC mass spectrometry [r²=0.93], and the LH assay with in vitro bioassay (r²=0.97) (Mulligan et al., Eur. J. Endocrinol. 141:257-266 (1999); Veldhuis et al., J. Clin. Endocrinol. Metab. 75:52-58 (1992); Krieg et al., Kidney Int. 58:569-574 (2000)). Infused GnRH concentrations were measured by validated RIA as described elsewhere (Veldhuis J D, Veldhuis N J, Keenan D M, Iranmanesh A; Am. J. Physiol. Endocrinol. Metab. 288:E775-E781, 2005; Veldhuis J D, Iranmanesh A, Clarke I A, Kaiser D L, Johnson M L; J. Neuroendocrinol 1:185-194, 1989).

A MLE-based deconvolution model, FIG. 7, was used to compute pulsatile and basal LH secretion rates and biexponential LH kinetics from individual 16-hours concentration-time series (Keenan et al., SIAM J. Appl. Math 61:934-965 (2000); Keenan et al., Am. J. Physiol. 275:R1939-R1949 (1998); Veldhuis et al., Am. J. Physiol. Endocrinol. Metab. 288:E775-E781 (2005); Keenan et al., Am. J. Physiol. 278:R1247-R1257 (2000); Erickson et al., J. Clin. Endocrinol. Metab. 89:4746-4754 (2004)). Pulsatile secretion can be the total amount of LH secreted in bursts segmented post hoc into before (12 hours) vs. after (4 hours) GnRH injection. A procedure can entail recursive estimation of the entire set of secretion and elimination parameters after each alternation (by probabilistic transitions) to a new putative pulse-time set. For example, see Keenan et al., J. Theor. Biol., 236: 242-255, 2005, incorporated in its entirely herein by reference.

A null hypothesis is that age over the span 18-80 years does not determine the degree to which free (protein-bound) Te concentrations enforce negative feedback on pulsatile LH. Secondary postulates can be that aging attenuates free Te's feedback on unobserved GnRH outflow (release and actions), and that free and bioavailable but not total Te concentrations can provide comparable feedback estimates.

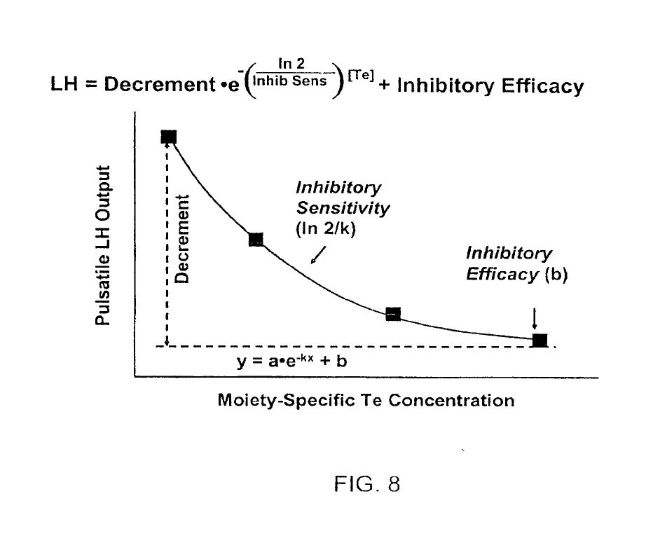

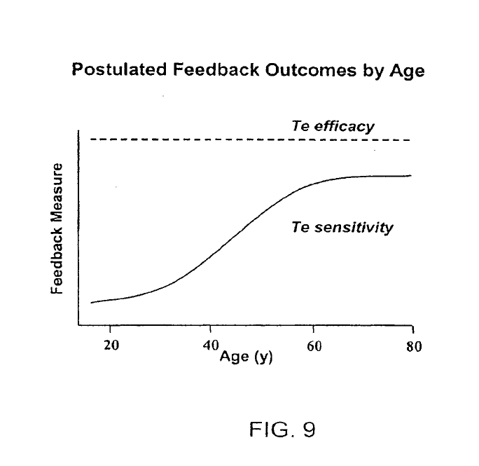

A reasonable initial approach can entail regression of both: (i) pulsatile LH output [sum of burst mass] prior to GnRH injection (IU/L/12 hours) exponentially on the mean (12-hours) free Te concentration (ng/dL) using data from all 4 infusion sessions in each individual, FIG. 8; and (ii) a set of 40 subject-specific estimates of free Te's feedback sensitivity (exponential coefficient determined in (i)) or efficacy (maximal inhibition determined in (i)) linearly on age, FIG. 9. A simultaneous (joint) regression formulation can be used to allow algebraically for intraindividual correlations in Te's feedback on LH (exponential function) and interindividual effects of age on either feedback measure (linear function) using data from all 40 subjects. An exponential function can be chosen to model asymptotic inhibition of LH output by Te and a linear function to model a hypothesized effect of age. A null hypothesis asserts that the linear slope of feedback on age is not negative. A slope is estimated at approximately 78 degrees of freedom (d.f.), given that the number of LH and Te observations in the exponential regression can be 160 (4×40); the number of ages in the linear regression is 40; and the number of parameters in the model is 122 (3×40+1 slope+1 intercept).

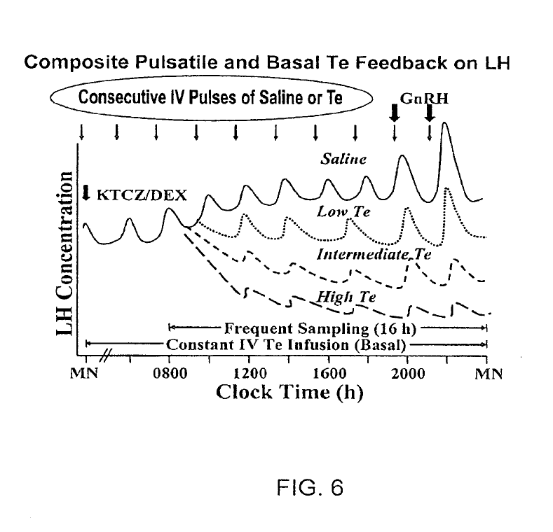

Estimates based on data described elsewhere (Zwart et al., J. Clin. Endocrinol. Metab. 82:2062-2069 (1997)) indicate that statistical power can exceed 90% to detect an effect of age that explains 33% or more of the variability in Te feedback sensitivity if 40 subjects are studied. The predicted slope±SD of feedback sensitivity regressed on age is (×10⁻³) 1.5±0.25. Pulsatile LH secretion (IU/L/min) can be expected to increase from 0.50 to 3.5 in the youngest and 0.65 to 2.0 in the oldest subjects in response to a free Te decrement (ng/dL) of 17 and 15, respectively, in the KTCZ/DEX paradigms of 7 mg vs. 1 mg Te addback (FIG. 6) (O'Brien, Biometrics 39:787-794, 1983). Because an a priori postulate is that age reduces feedback sensitivity, a test of significance can be one-tailed; and because both sensitivity and efficacy can be examined, the experiment-wise critical value is P≦0.025. Secondary queries (unpowered) are whether age decreases Te's inhibition of (a) exogenous GnRH-stimulated LH secretion, (b) the frequency of LH pulses (number per 12 hours), and (c) the regularity (approximate entropy) of LH release.

One objective of the method can be to estimate the potency, efficacy and sensitivity of implicit nonlinear dose-response functions linking time-varying (rather than 8-hours mean) free Te concentrations to observed LH secretory bursts and to unobserved (reconstructed) GnRH pulses jointly by way of GnRH→LH feedforward and Te→GnRH/LH feedback under deterministic and stochastic allowances.

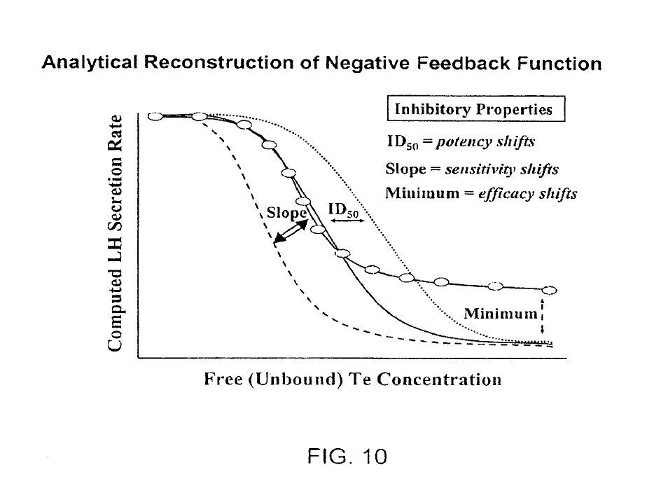

According to basic principles of ligand-receptor interactions, properties of a dose-response interface can be sensibly represented by a monotonic, cooperative 4-parameter logistic function defined by sensitivity, efficacy, potency and baseline (below). Feedback estimation can require formulating free Te's concentration-dependent inhibition of LH secretion using all LH and Te measurements in each subject (data sampled every 10 min for 12 hours on four occasions or (12×6+1)(4)=292 paired estimates of LH and Te). To this end, sample-discretized LH secretion rates can be represented as a negative logistic function of antecedent (30-min integrated) free Te concentrations, FIG. 10. The data set comprises 581 (292+289) observations and the parameter set 19 dependent variables, viz.: (a) subject-specific LH secretory-burst shape (three-parameter generalized Gamma probability density) and elimination kinetics (three-parameter biexponential); (b) session-selective nonpulsatile (basal) secretion rates (4 levels), random effects on burst mass (4) and experimental error (1); and (c) 4 feedback dose-response parameters.

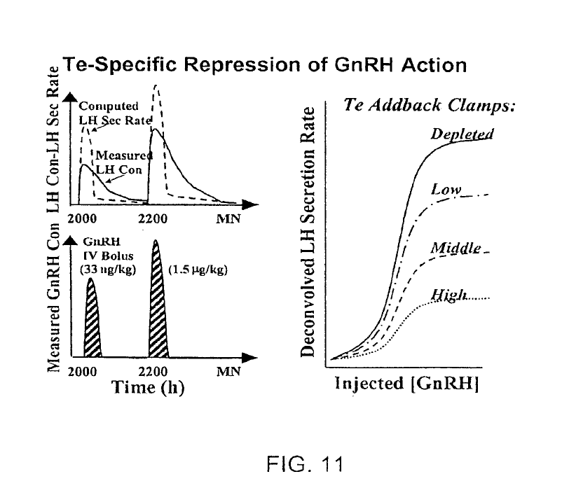

A second step can be to verify estimation of unobserved GnRH outflow by constructing free Te's feedback on exogenous GnRH-stimulated LH secretion. Data can include sample-discretized LH secretory rates over four hours and frequently (1 to 2.5-min) measured concentrations of injected GnRH (first and second-phase half-lives, 3-7 and 35-50 min), (Handelsman et al., Endocr Rev 7:95-105 (1986)) (FIG. 11). A GnRH→LH feedforward (concentration-secretion) dose-response function can be evaluated first at each experimentally imposed Te stratum in each subject, as validated initially for LH→Te feedforward estimation. Resultant estimates can provide starting values to construct a three-dimensional GnRH-LH-Te dose-response surface using all data simultaneously in each subject. A surface can be defined jointly by one analogous logistic function embodying (injected) GnRH's concentration-dependent stimulation of LH secretion (GnRH→LH feedforward) and another four-parameter logistic function representing (calculated) free Te's concentration-dependent inhibition of GnRH-induced LH secretion (Te→GnRH/LH feedback).

Data in each person can include (10-min) discretized LH secretion rates and (30-min integrated) free Te concentrations over 4 hours (together, N=46 measures per session) and 28 GnRH concentrations per session (totaling in 4 sessions 296 observations). Estimation of the (8-parameter) dose-response surface in each subject can be accomplished at greater than 288 d.f. An overall result is 40 (subject-specific) estimates of the efficacy, potency and sensitivity of free Te's inhibition of GnRH-stimulated LH secretion. Each feedback coefficient can then be regressed linearly on age. A null hypothesis is that the slope on age is not negative at one-tailed P<0.0167 (penalized for assessment of three feedback coefficients). The technical point of evaluating injected GnRH pulse contours is to verify estimation of GnRH→LH and Te→GnRH/LH interface parameters based upon direct measurements of all three signals over time. The low and high doses of GnRH can be chosen as one-half (potency) and maximally (efficacy) stimulating.

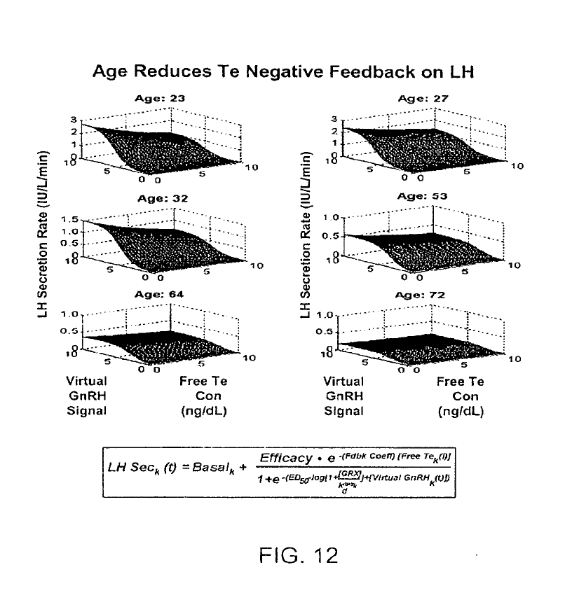

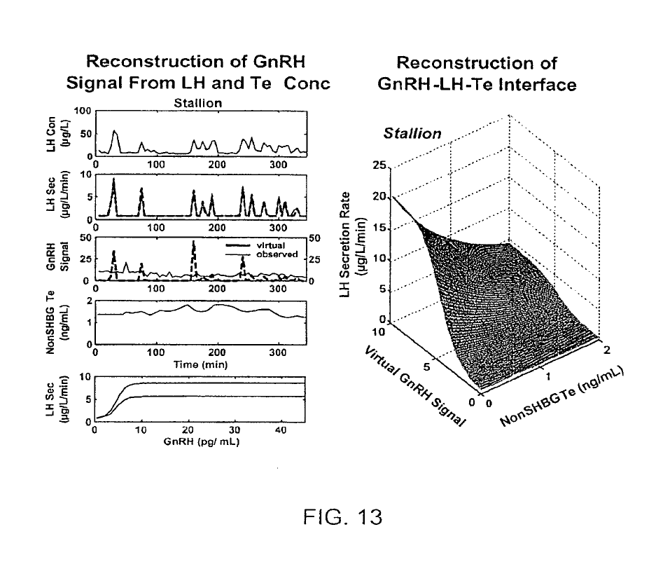

A third estimation step can utilize the 12-hours LH and Te concentration time series obtained prior to GnRH injection in each subject to reconstruct pulsatile free Te's feedback on endogenous (noninjected) GnRH-driven LH secretion. To ensure statistical validity, three of the unobserved (virtual) GnRH signals, GnRH→LH feedforward and Te→GnRH feedback parameters can be estimated simultaneously in any one subject using the combined data from all four infusion sessions. An algebraic formulation is given in FIG. 12, which illustrates pilot estimates of GnRH-LH-Te dose-response surfaces in six normal men. Here, four strata of Te availability (designated k) were created by graded competitive antagonism of GnRH-receptor signaling. Empirical assessment of model performance was by frequent (five-min) measurements over 12 hours of GnRH and LH in the pituitary sinus and (nonSHBG-bound) Te in the jugular vein of a conscious unrestrained stallion, FIG. 13. The left side compares directly measured and analytically estimated LH, GnRH and Te time series with estimates of GnRH→LH based upon the minimum and maximum measured Te concentration in that sampling session; and the right side presents model-based joint reconstruction of GnRH's concentration-dependent stimulation and Te's concentration-dependent inhibition of pulsatile LH secretion.

Increasing age over the span 20-80 yr can: (a) blunt intermittent feedback by experimentally controlled Te pulses on pulsatile LH output (summed LH secretory-burst mass over 12 hours); (b) reduce the estimated amount of hypothalamic GnRH released per pulse; and (c) not impair stimulation of LH secretion by exogenous GnRH. In addition to the foregoing particular findings, success of the method can be defined by an evolution of a generic construct for quantifying nonlinear time-varying negative-feedback control of un-manipulated neurohormone outflow noninvasively without infusing agonists or antagonists (below).

Bioavailable (nonSHBG-bound), albumin-bound, SHBG-bound and total Te concentrations can also correlate with certain tissue effects of androgens in the brain, pituitary gland, prostate and liver (Mendel, Endocr. Rev. 10:232-274, 1989). The present analyses will be able to explore, secondarily, which biological fractions of plasma Te best predict in vivo feedback on the human hypothalamo-pituitary unit.

How aging impacts negative feedback by estradiol is currently not known, although this sex steroid can repress both GnRH and LH secretion (Veldhuis et al., J. Clin. Endocrinol. Metab. 82:1248-1254 (1997), Schnorr et al., J. Clin. Endocrinol. Metab. 86:2600-2606 (2001); D'agata et al., Acta Endocrinol. (Copenh) 97:145-149 (1981); Bhatnagar et al., J. Steroid Biochem. Mol. Biol. 41:437-443 (1992); Naftolin et al., Am. J. Obstet. Gynecol. 147:491-496 (1983); Urban et al., J. Androl. 12:27-35 (1991); Urban et al., J. Clin. Endocrinol. Metab. 72:660-668 (1991)). The present model structure, if verified for Te, could provide a platform for addressing this basic query and analogous questions in the female gonadal axis.

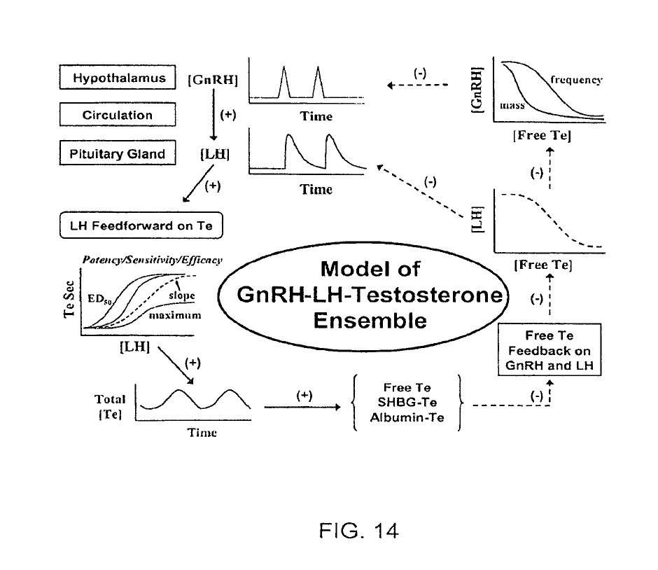

This document describes some prerequisites for estimating unobserved regulatory adaptations in vivo, comprising: (i) a verifiable analytical construct to quantify time-varying interactions in the undisturbed host; and (ii) a suitable empirical paradigm of intermittent feedback signaling to establish clinical validity. Technical goals can include reconstructing analytically each of: (i) pulsatile and basal LH secretion rates; (ii) time-varying free, bioavailable and total Te concentrations; (iii) elimination kinetics of Te and LH; (iv) unobserved GnRH signals; and (v) nonlinear dose-response functions representing how GnRH pulses drive, and how Te pulses repress, LH secretion. These key connections can be shown schematically in FIG. 14.

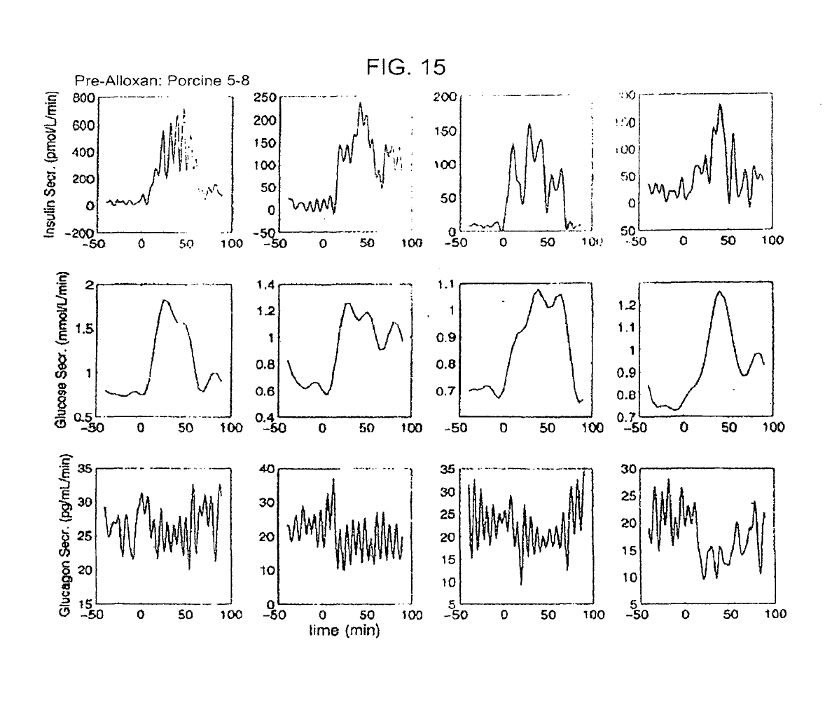

FIG. 15 shows model estimates of insulin, glucose and glucagon secretion rates (in 4 pigs sampled frequently, pre-alloxan) using model equations [1]-[8]. Data were obtained as described in Kjems L L, Kirby B M, Welsh E M, Veldhuis J D, Straume M, McIntyre S S, Yang D, Lefebvre P, Butler P C, 2001 (ibid). Decrease in beta-cell mass leads to impaired pulsatile insulin secretion, reduced postprandial hepatic insulin clearance, and relative hyperglucagonemia in the minipig.

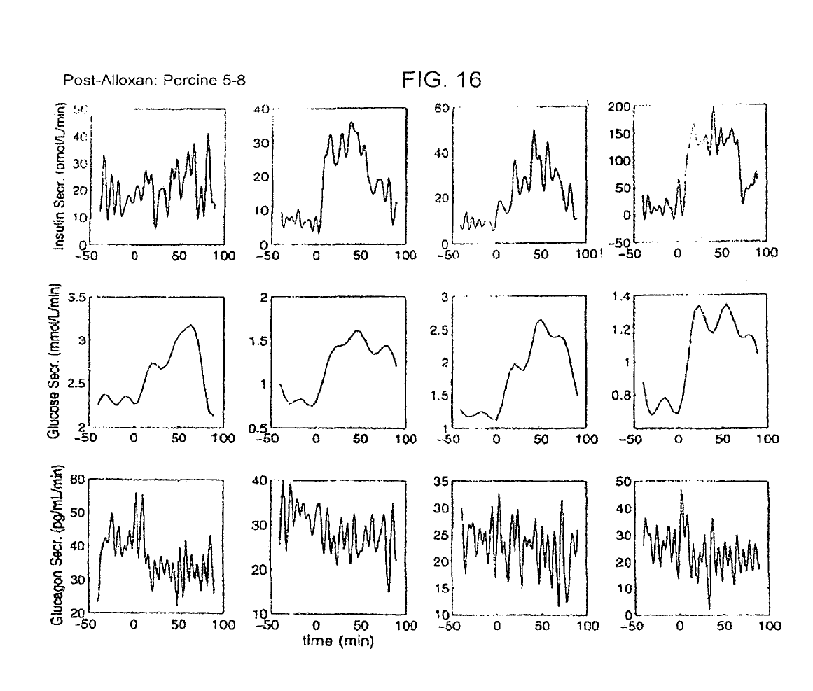

FIG. 16 shows new estimates in the same 4 pigs (as FIG. 15) but after alloxan-induced diabetes in the animal.

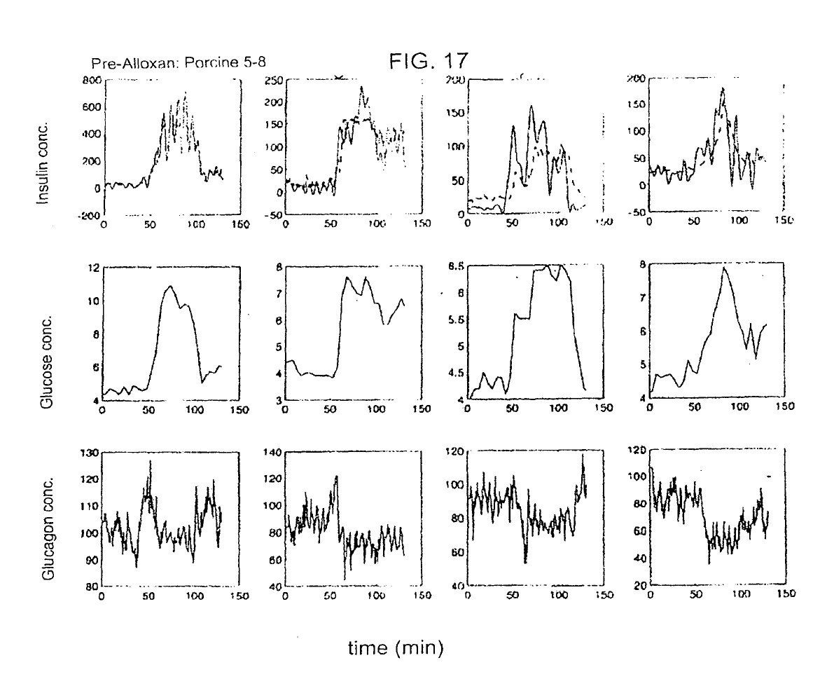

FIG. 17 shows model estimates of insulin, glucose and glucagon concentrations (interrupted lines) in 4 separate pigs (intact, pre-alloxan) using model equations [1]-[8 ].

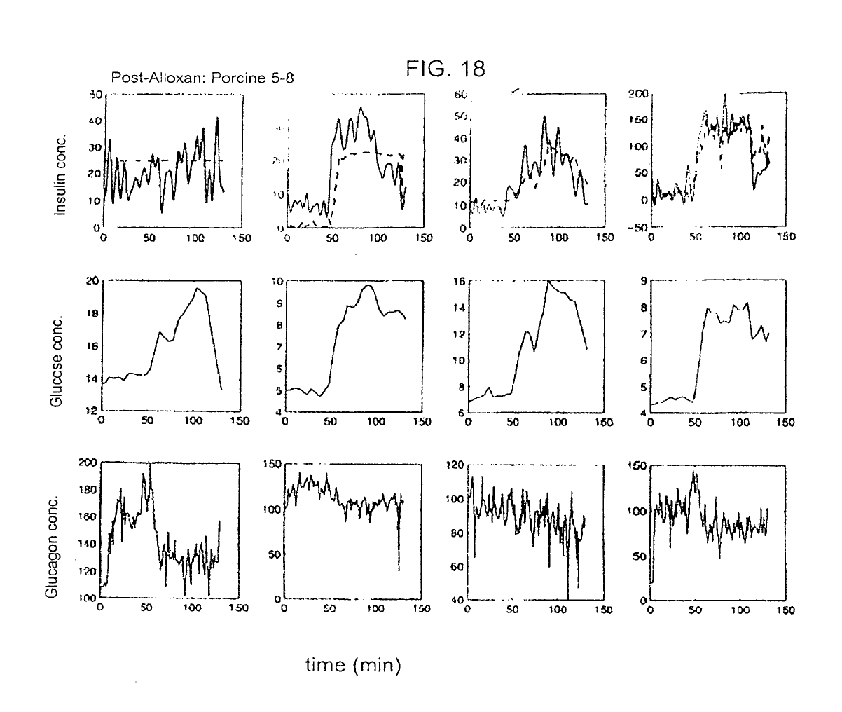

FIG. 18 shows new estimates in the same 4 pigs (as FIG. 17) after diabetes was induced by alloxan.

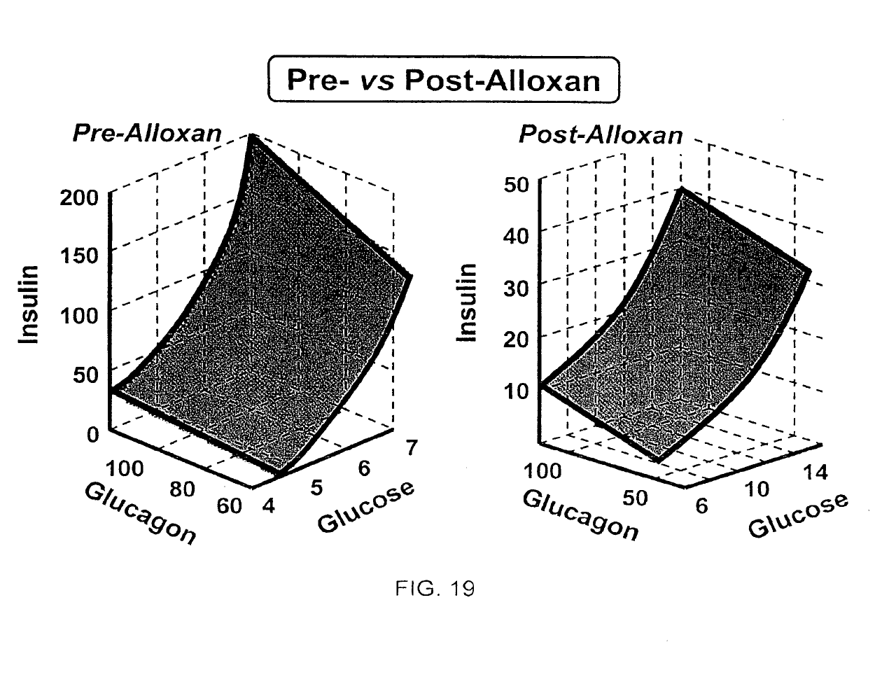

FIG. 19 shows model-based calculations using equations [1]-[8] and measurements from pig #7 (from FIGS. 15-18) of the surface defining how increased glucose elevates insulin and glucagon concentrations less in the post-alloxan model.

Neuroendocrine systems can communicate via pulsatile signals, which convey distinct information to target tissues. The timing, shape and amplitude of discrete pulses can be dictated by intermittent feedforward and feedback inputs, as typified by hypothalamic effectors that direct the synthesis, storage and release of anterior-pituitary hormones (Urban et al., 1988; Evans et al., 1992; Giustina & Veldhuis, 1998). Thus, the mechanisms that govern neurohormone pulsatility can also mediate integrative and regulatory control of an ensemble axis (Pincus et al., 1996; Keenan et al., 2001; Keenan et al., 2004). However, the interconnected nature of endocrine signaling creates a major investigative problem; viz., manipulating or isolating any system component would definitionally disrupt physiological feedforward and feedback interactions. This impasse has motivated the development of noninvasive methods to quantify rapidly adaptive changes in secretion patterns.

Protein hormones are encapsulated within secretory granules, which diffuse toward and dock at the cellular membrane (Aryan et al., 1991). A pool of exocytotic vesicles permits immediate release, and granule replenishment allows for time-delayed secretion, resulting in a skewed burst-like secretion waveform (Redekopp et al., 1986; Clarke et al., 2002; Veldhuis et al., 2002). In addition, a fraction of synthesized molecules is lost from the cell by basal or continuous release (Veldhuis et al., 2002; Aryan et al., 1991). Steroidogenic glands such as the adrenal, ovary and testis also secrete signals intermittently under pulsatile drive by the cognate trophic hormone, albeit with a higher basal component (Urquhart & Li, 1968; Keenan & Veldhuis, 1998; Foresta et al., 1997; Keenan et al., 2001; Keenan & Veldhuis, 2004). After secretion, molecules undergo random diffusion in tissue fluids and blood, intravascular advection (linear flow) and irreversible elimination from the circulation. Accordingly, valid appraisal of neuroendocrine dynamics requires simultaneous estimation of all three of basal release, pulsatile secretion and elimination processes in the presence of random experimental variability.

From an analytical perspective, determination of intercorrelated secretion and elimination rates in a pulsatile model requires reliable initial estimation of secretory-burst location and number (Veldhuis et al., 1987; Veldhuis & Johnson, 1995). However, in most neuroendocrine settings the timing of secretory events (pulse-onset times) appears to be random (Keenan & Veldhuis, 1997; Keenan & Veldhuis, 1998; Keenan et al., 2000; Butler et al., 1986; Camproux et al., 1994). One analytical strategy therefore is to estimate all three of pulse times and secretion and elimination parameters jointly in a statistically justified fashion. The approach entails a Bayesian formulation (a probability distribution is constructed on the parameters, based upon the data), which yields probabilistic estimates of secretory and kinetic properties underlying any particular individual time series.

Methods

A. Overview of Methods for Pulse Detection

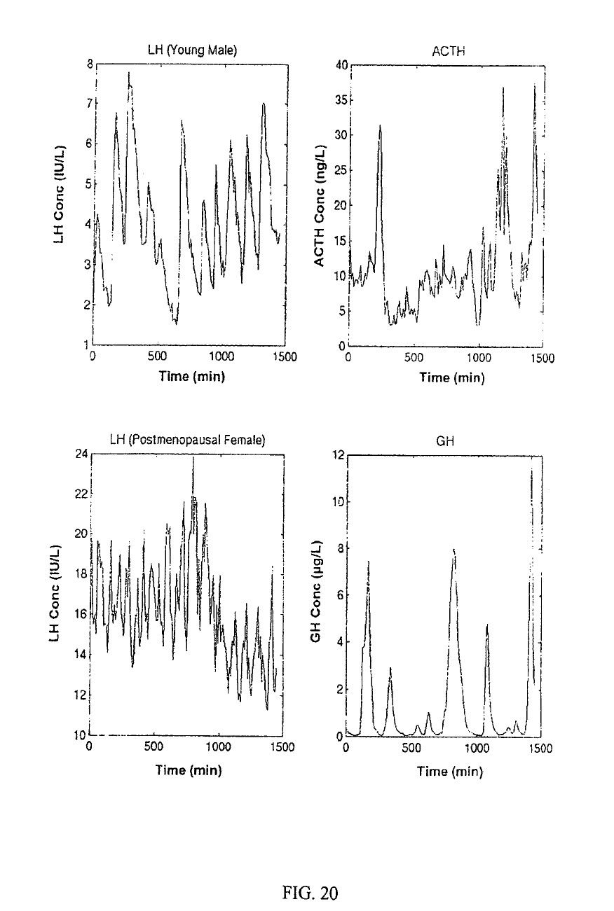

The experimentalist requires an automated procedure by which to simultaneously detect pulse times and estimate secretion/kinetic features. To this end, consider the four pituitary-hormone concentration time profiles displayed in FIG. 20: Luteinizing hormone (LH) in a young man, adrenocorticotropin hormone (ACTH) in a woman, LH in a postmenopausal woman and growth hormone (GH) in a man, which were obtained earlier (Keenan & Veldhuis, 2004; Keenan et al., 2003; Keenan & Veldhuis, 2003). In each case, discrete blood sampling (i.e., the withdrawal lasting 5-10 sec) was performed every 10 min for 24 hr. [Integrative sampling (continuous withdrawal) yields a time-integrated or flattened version of the impulse, thereby underestimating absolute peak values and overestimating interpulse valleys.] If the set (Y₁, Y₂, . . . , Yₙ) denotes observed hormone concentrations, n would be 145 for 10-min sampling over 24 hr. As an example of the objectives of the present methods, consider the question of what is the total 24 hr amount of pulsatile LH secretion (i.e., excluding basal) for the above young male, having only observed the 24-hr LH concentrations. One would like to find values (a,b) so that, for a specified probability, e.g., 0.95, one can make the following probability statement:

P(a<24-hr Pulsatile LH Secretion(IU/L/day)<b|(Y₁, Y₂, . . . , Yₙ))=0.95 [9]

In Eq. [20] (below), the present methods give an explicit solution to this.

(a) Prior Strategies

Various methods have been proposed for detecting pulse times, some of which also estimate hormone secretion and/or kinetics. Mauger, Brown and Kushler (1995) summarized many proposed techniques, and compared performance on simulated data (Mauger et al., 1995). These authors distinguish between criterion-based and model-based methods. The former strategy uses a test statistic to identify a pulse over confounding experimental variability, whereas the latter adopts a statistical model and estimates its parameters. Pulse-evaluation procedures classified as criterion-based methods include those suggested by Santen and Bardin [1973] (Santen & Bardin, 1973), Goodman and Karsch [1980] (Goodman & Karsch, 1980), Merriam and Wachter [1982] (Merriam & Wachter, 1982), Clifton and Steiner [1983] (Clifton & Steiner, 1983), Veldhuis and Johnson [1986] (Veldhuis & Johnson, 1986), Oerter, Guardabasso and Rodbard [1986] (Oerter et al., 1986), Van Cauter et al. [1981] (Van Cauter, 1981), and Munson and Rodbard [1989] (Munson & Rodbard, 1989). Model-based approaches encompass those developed by Veldhuis and Johnson [1987] (Veldhuis et al., 1987), O'Sullivan and O'Sullivan [1988] (O'Sullivan & O'Sullivan, 1988), Diggle and Zeger (Diggle & Zeger, 1989), Kushler and Brown [1991] (Kushler & Brown, 1991), Veldhuis and Johnson [1992] (Veldhuis & Johnson, 1992), and Veldhuis and Johnson (Veldhuis & Johnson, 1995). Since that review, Keenan and Veldhuis [1997] proposed a model-based approach, which seeks to incorporate physiological principles of regulated hormone synthesis, accumulation, release and elimination (Keenan & Veldhuis, 1997; Keenan & Veldhuis, 1998; Keenan et al., 1998; Keenan et al., 2000; Keenan et al., 2001; Keenan et al., 2004). This construction was, however, conditional on valid peak identification (Keenan & Veldhuis, 2003; Keenan & Veldhuis, 2004).

(b) General Objectives

To our knowledge, noninvasive joint estimation of pulse number and location, basal and pulsatile hormone secretion and nonequilibrium kinetics has not been accomplished. The present work addresses this goal.

As general guidelines, we recognize several objectives of pulse evaluation. First, the algorithm should be adaptable. For example, pertinent system-level feedforward and feedback inputs and any available knowledge of secretion and elimination properties might be incorporated into the overall formulation later without great difficulty. Second, the structure should be relevant to the physiological problem. Third, implementation should be reproducible, systematic and automated (not requiring human input). In particular, the decision-making procedure must probabilistically add or remove a justifiable pulse and define its presumptive location in the time series. And, fourth, the process of recursive estimation of secretion and elimination parameters must proceed jointly with pulse-time assignments according to appropriate statistical criteria. Based upon the foregoing expectations, the resultant idealized platform would be both analytical (model-assisted) and statistical (criterion-defined).

B. Model of Hormone Concentrations for a Given Set of Pulse Times, ℑₘ

Assuming that pulse-onset times are given by ℑₘ={T¹, T² . . . , T^{m}}, Keenan and Veldhuis give a model to estimate secretory dynamics conditional on pulse times (Keenan & Veldhuis, 1998; Keenan et al., 2000; Keenan et al., 1998). The primary components are basal and pulsatile secretion, a flexible secretory-burst shape, random effects on burst mass, biexponential elimination kinetics and combined experimental uncertainty in sample collection, processing and assay. We suppose that M, the amount of hormone secreted in the j^{th} burst (mass per unit distribution volume), is the sum of a finite amount of minimally available stores, a linear function of hormone accumulation over the preceding interpulse interval, and a random effect allowing for biological variability in individual burst mass:

M^{j}=η₀+η₁×(T^{j}−T^{j-1})+A^{j} [10]

where η₀ is minimal releasable hormone, η₁ a linear coefficient operating on mass accumulated over the preceding interburst interval, T^{j}−T^{j-1}, and A^{j} a random effect (Keenan et al., 2001). The mass contained in any given burst, M^{j}, is released in the time-profile of an adaptable (hormone-, subject- and condition-specific) waveform. The waveform (evolution of instantaneous secretion rate over time) is homogeneous within any given time series and represented via a three-parameter generalized Gamma function with units of mass released per unit time (min) per unit distribution volume (L),

$$ψ ( s ) ∝ {s}^{{β}_{1} {β}_{3} - 1} {e}^{- {( s / {β}_{2} )}^{{β}_{3}}} , s \geq 0. \qquad [ 11 ]$$

The beta parameters allow variability in the rates of onset (β₁β₃), peakedness (β₃) and dissipation (β₃/β₂) of the secretory event. Members of the Gamma family of probability distributions are normalized to unit area, and therefore this “shape” function is independent of size (mass) of the burst. Gamma densities can also approximate symmetric waveforms (e.g., the 2-parameter Gaussian function).

The amount of hormone secreted in a burst is the product of the mass (Eq. [10]) and the normalized psi function (Eq. [11]). The total secretion rate, Z, is the sum of time-invariant (constitutively basal) hormone release, β₀, and pulsatile secretion.

$$Z ( r ) = {β}_{0} + {\sum }_{{T}^{j} \leq r} {M}^{j} ψ ( r - {T}^{j} ) \qquad [ 12 ]$$

Earlier it was shown that at any instant in time, t, the hormone concentration, X(t), sampled at a given point in the circulation, x, can be described by (coupled) differential equations defining total secretion and overall elimination. The analytical solution of this representation is a summed biexponential function:

$$X ( t ) = ( \begin{matrix}a {e}^{- {α}_{1} t} + \\ ( 1 - a ) {e}^{- {α}_{2} t}\end{matrix} ) X ( 0 ) + {\int }_{0}^{t} ( \begin{matrix}a {e}^{- {α}_{1} ( t - r )} + \\ ( 1 - a ) {e}^{- {α}_{2} ( t - r )}\end{matrix} ) \times Z ( r ) d r \qquad [ 13 ]$$

where a is the (amplitude) proportion of rapid to total elimination, Z(r) the secretion rate, and X(0) the starting hormone concentration. In this formulation, the rate constants of fast and slow elimination primarily embody the respective contributions of molecular diffusion (random motion) and advection (linear flow) in blood (α₁) and irreversible loss (α₂) from plasma (Keenan et al., 2004).

Based upon a discrete-time sampling Δt=tᵢ−tᵢ₋₁, i=1, . . . , N, we define the series of observed hormone concentrations, Yᵢ, as jointly due to elimination, observational error, εᵢ, and the discrete-time secretion rate, Zᵢ:

$${Y}_{i} = X ( {t}_{i} ) + {ɛ}_{i} , {Z}_{i} = Z ( {t}_{i} ) , i = 1 , … , n , \\ U = ( \begin{matrix}{u}_{11} & {u}_{12} & … & {u}_{1 m} \\ {u}_{21} & {u}_{22} & … & {u}_{2 m} \\ ⋮ & ⋮ & ⋮ & ⋮ \\ {u}_{n 1} & {u}_{n 2} & … & {u}_{n m}\end{matrix} ) , {u}_{ij} = \{ \begin{matrix}0 , & if {T}^{j} \geq i \\ ψ ( i - {T}^{j} ) , & if {T}^{j} < i ,\end{matrix}$$

$$Y = ( \begin{matrix}{Y}_{1} \\ {Y}_{2} \\ ⋮ \\ {Y}_{n}\end{matrix} ) , Z = ( \begin{matrix}{Z}_{1} \\ {Z}_{2} \\ ⋮ \\ {Z}_{n}\end{matrix} ) , A = ( \begin{matrix}{A}^{1} \\ {A}^{2} \\ ⋮ \\ {A}^{m}\end{matrix} )$$

For a discrete time sampling Δt, for j=1, 2: \tilde{α}^{(j)}=(1−αⱼΔt),

$${R}_{{\tilde{α}}^{( j )}} = ( \begin{matrix}1 & 0 & 0 & … & 0 & 0 \\ - {\tilde{α}}^{( j )} & 1 & … & … & 0 & 0 \\ 0 & - {\tilde{α}}^{( j )} & 1 & … & 0 & 0 \\ ⋮ & ⋮ & ⋮ & ⋱ & ⋮ & ⋮ \\ 0 & 0 & 0 & … & - {\tilde{α}}^{( j )} & 1\end{matrix} ) \\ and {R}_{{\tilde{α}}^{( 1 )} , {\tilde{α}}^{( 2 )}} = a {R}_{{\tilde{α}}^{( 1 )}} + ( 1 - a ) {R}_{{\tilde{α}}^{( 2 )}} \\ θ = ( γ , σ ) = ( ( {β}_{0} , {\tilde{α}}^{( 1 )} , {\tilde{α}}^{( 2 )} , {η}_{0} , {η}_{1} , {β}_{1} , {β}_{2} , {β}_{3} ) , ( {σ}_{A}^{2} , {σ}_{ɛ}^{2} ) ) \\ Y = υ + {R}_{{\tilde{α}}^{( 1 )} , {\tilde{α}}^{( 2 )}}^{- 1} Z + {R}_{{\tilde{α}}^{( 1 )} , {\tilde{α}}^{( 2 )}}^{- 1} U ( γ ) A + ɛ , \qquad [ 14 ]$$

where υ depends on the initial condition. Assuming independent Gaussian models for the vectors ε and A, conditioned on the pulse-time set ℑₘ, yields a Gaussian log-likelihood function: l_{ℑ}_{ₘ}(θ|Y), or equivalently, the minus log-likelihood function:

−l_{ℑ}_{ₘ}(θ|Y) [15]

(Most optimization software, as a matter of convention, formulate problems as those of minimization.) Minimizing the last function with respect to θ allows a maximum-likelihood estimate (MLE) of θ. Yang [1997] and Keenan, Veldhuis and Yang [1998] (Keenan et al., 2000) presented asymptotic results justifying this MLE method, conditional on the pulse times ℑₘ={T¹, T² . . . , T^{m}}, for m (=# random effects, i.e. pulses) sufficiently small relative to the number of observations n. In this earlier approach, potential drawbacks included: (1) the required first-stage estimation of pulse times ℑₘ, which are then assumed to be fixed; and (2) interpretation of the unknown secretion and kinetic parameters as particular and fixed (rather than probabilistic) values for any given data set. The accompanying Bayesian algorithm rectifies these two limitations.

The concept adopted below is that, for a given set of pulse times and secretion/elimination parameters, the estimation process must determine whether to add a new pulse time, consolidate two into one, or remove one. Whatever the choice, estimation must be redone on the complete continuous parameter space [Eq. 14] to test for an improvement in overall fit. Valid statistical alternation of discrete (pulse times) and continuous (secretion/elimination) parameter estimation is necessary in view of their interdependence. The objective is a probabilistic interpretation of the pulse number and parameter estimates for any single neurohormone time series.

C. Constructing (Data-Dependent) Sets of Candidate Pulse Times

The fact that the number and locations of the pulse times is unobserved, introduces an important complexity. If one observed the pulse times (locations and their number), then one could model the pulse frequency and even the regularity of the pulsing; this was done in Keenan and Veldhuis [2001] (Keenan & Veldhuis, 2001), using a Weibull renewal process model. However, not knowing the pulse times places the problem beyond computational capability (e.g., using a conditioning/unconditioning approach). An alternative is to construct a data-dependent collection of putative pulse time sets.

The pulse-estimation component is based on a methodology proposed initially in computer vision and image-processing technologies to detect boundaries of objects (Alvarez, Lions & Morel, 1992). The rationale is that presumptive boundaries define points of more rapid change, just as the onset of a pulse marks more rapid change. Selective smoothing addresses this goal by (definitionally) imposing little change at points of very rapid increase (pulse-onset times) and greater smoothing on points of less rapid increase (nadirs), thereby removing small variations that confound pulse detection.

The first stage of selective smoothing identifies all nadirs as potential pulse times. Suppose that the time series Y has N local minima, each defined by a first-derivative sign change from negative to positive. As smoothing proceeds in algorithmic time, one of the local minima is smoothed away and the resulting new set of local minima will comprise N−1 points. If the algorithm ran ad infinitum, Y would be smoothed to a constant mean value. In practice, some pulses evolve with a “stuttering” onset, wherein an initial slight increase precedes a large rapid increase; in the present method such points are not excluded from putative pulse-time sets. For pragmatic implementation, smoothing evolves for some pre-specified number of algorithmic cycles or until some pre-specified minimal number (e.g., p) of pulse times. The results are sets of decreasing numbers of provisional pulse-onset times:

ℑ={ℑ_{N}, ℑ_{N-1}, . . . , ℑₚ} [16]

To eliminate the need for human decision-making in the selection of pulse times, the algorithm determines where and in which order to remove conjectured pulses. For concentrations given by {Y(t), 0≦t≦1}, where t represents observational time (e.g., min over a day), the following iterative algorithm is used based upon: (a) an initial condition; (b) a Dirichlet boundary condition; and (c) a selective-smoothing equation:

$$\begin{matrix}u ( t , 0 ) = Y ( t ) , 0 \leq t \leq 1 , ( Initial Condition ) & \\ u ( 0 , s ) = Y ( 0 ) , u ( 1 , s ) = Y ( 1 ) , ( \begin{matrix}Dirichlet Boundary \\ Condition\end{matrix} ) & \\ 0 \leq s \leq S , & \\ \frac{\partial u ( t , s )}{\partial s} = g ( {( \frac{\partial u ( t , s )}{\partial t} )}_{+} ) \frac{{\partial }^{2}}{\partial {t}^{2}} u ( t , s ) , & [ 17 ] \\ g ( x ) = {C}_{1} / ( 1 + {( x / {C}_{2} )}^{2} ) , & \\ 0 < x < {C}_{2} , {C}_{1} > 0 , {C}_{2} > 0. ( Selective Smoothing Equation ) & \end{matrix}$$

A constant coefficient, g, in [Eq. 17] would yield the classical linear diffusion (or heat) equation. However, in the present case, the diffusion coefficient g(•) is a function of the derivative of concentration on time. When the derivative is large in absolute value, smoothing at that point (x) is minimal, and, conversely. The construction distinguishes between positive and negative derivatives (upstrokes and downstrokes in the data). The smoothing process continues for 0≦s≦S, where s refers to algorithmic time.

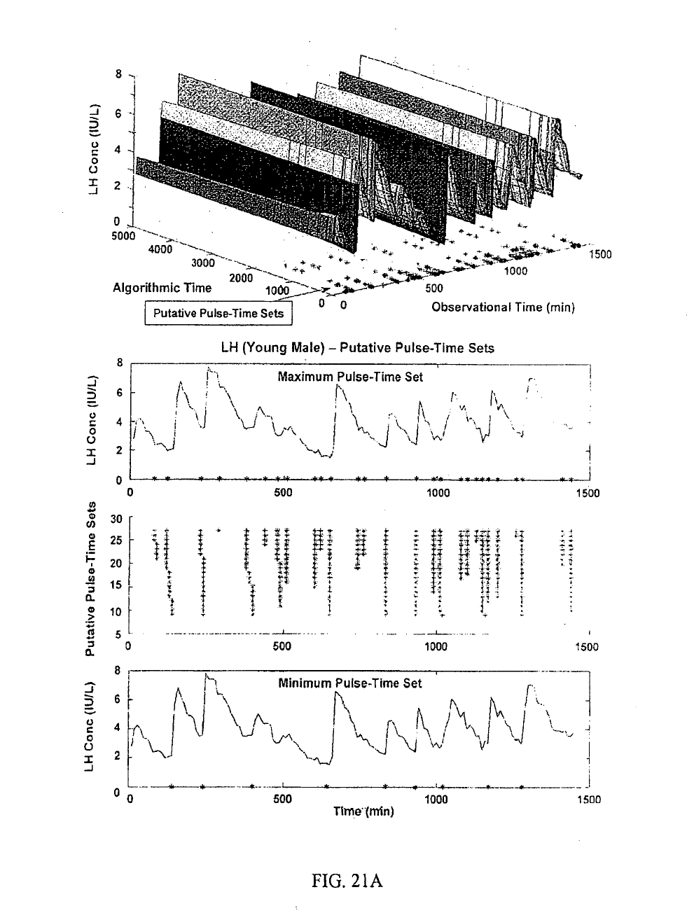

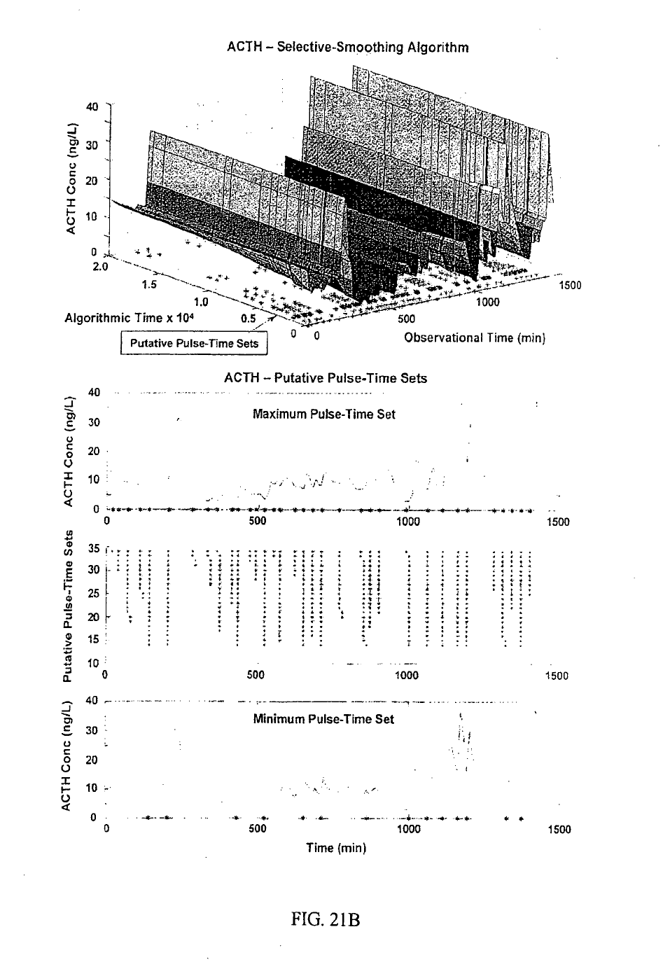

FIG. 21A illustrates the output of the selective-smoothing algorithm applied to an LH concentration time series observed in a young man (FIG. 1, left top). The bottom three panels show LH concentration plots and sequential pulse-time sets: ℑ={ℑ_{N}, ℑ_{N-1}, . . . , ℑₚ}, wherein pulses are identified individually by asterisks. The upper and lower boxed LH profiles present pulse-onset times for the maximum and the minimum number of events in the pulse-time sets shown. The top 3-dimensional plot represents the surface u, given by Eq. [17], wherein cross-sections (for each fixed algorithmic time, s) represent smoothed versions of the original concentration profile unfolding over observational time, t. During the algorithmic window displayed here, the number of presumptive pulses decreases from N=27 top=9. FIG. 21B gives analogous estimates of pulse onsets for an ACTH time series (FIG. 1, right top). In the discretization of Eq. [17] for FIGS. 21A and 21B (10 min sampled data (⅙ hr)), the following assumptions were made: Δx=(⅙), Δt=(⅙)², C1=0.3, C2=0.01. From experimentation, the space and time step choice is less important than the values of C1 and C2. (Justification for the discretization is given in (John, 1982, Chapter 7.2)).

D. Combined Pulse Time and Secretion/Elimination Modeling

The present algorithm reflects the biology of pulsatile hormone secretion and illustrates the application of several diverse applied mathematical methods (partial differential equations, stochastic processes and Bayesian statistics) to the detection, based upon only concentration data, of the underlying (unobserved) pulse times. The flow and objectives of the algorithm are quite natural and can be easily grasped with only an intuitive understanding of these mathematical methods, as presented below. Moreover, the general approach of the algorithm has become a standard way to handle complex Bayesian calculations. For mathematical clarity a Summary of Algorithmic Flow gives a step-by-step elucidation of the procedure.

In the present Bayesian approach, an a priori (prior) probability distribution is placed on the parameter space: Θ×ℑ, where ℑ={ℑ_{N}, ℑ_{N-1}, . . . , ℑₚ} in Eq. [16] and Θ denotes the parameter set in Eq. [14]. The prior density on Θ×ℑ is assumed to be a product: π×λ, with a uniform prior on Θ, π(θ)∝ to a Constant. The prior on ℑ is the Akaike Information Criterion (AIC) penalty for the number of pulse times m, λ(ℑₘ)∝exp(−m). The theoretical justification for this method does not depend on the choices of priors. Measured data Y=(Y₁, Y₂, . . . , Yₙ)′ are then incorporated via a likelihood function (Eq. [15]), resulting in the a posteriori (posterior) probability distribution on Θ×ℑ:

P((θ, ℑₘ)|(Y₁, Y₂, . . . , Yₙ)), (θ, ℑₘ)εΘ×ℑ [18]

The objective is to develop a procedure to simulate from this posterior distribution, which circumvents any direct probability calculation (e.g., by high-dimensional integration). The analytical difficulty with the resulting posterior distribution of Eq. [18] is its enormous complexity. The present algorithm converts the complex analytics into a procedure, which is easy to describe but computationally intensive. Specifically, for a fixed pulse time set ℑₘ, let h_{ℑ}_{ₘ}(θ|Y)∝−(l_{ℑ}_{ₘ}(θ|Y)×ln π(θ)), where π is the prior probability density on Θ, and for which the normalization is such that

$${e}^{- {h}_{{𝒯}_{m}} ( θ \vert Y )}$$

is the posterior probability density on Θ. The essence of this method is to simulate from the posterior distribution by running a stochastic differential equation until the algorithmic (or iterative) time t, is large:

dθₜ=−∇h_{ℑ}_{ₘ}(θₜ|Y)dt+\sqrt{2}dBₜ, [19]

where Bₜ is standard Brownian motion (and the \sqrt{2} is a consequence of Brownian motion). This procedure is called stochastic relaxation [or Markov Chain Monte Carlo, MCMC], which is performed on the parameter set Θ in combination with probabilistic transitions within the collection of pulse sets ℑ. The result is an algorithm for generating samples from the joint posterior (Eq. [18]) on Θ×ℑ.

To place the method in perspective, one can minimize −h_{ℑ}_{ₘ}(θ|Y) (in θ, for ℑₘ fixed) by following the gradient flow: dθₜ=−∇h_{ℑ}_{ₘ}(θₜ|Y)dt, which would yield a local minimum; multiple starting points could aid in finding a global minimum. An alternative, which probabilistically avoids getting caught in a local minimum, is simulated annealing. [Parameter evaluation in this case follows a stochastically perturbed (noisy) gradient flow: dθₜ=−∇h_{ℑ}_{ₘ}(θₜ|Y)dt+c(t)dBₜ, with gradually decreasing noise: c(t)∝\sqrt{2}/ln(1+t). This would lead one to the mode of the posterior distribution on Θ. However, because of the restriction of logarithmically decreasing noise, c(t), simulated annealing is advantageous only in high-dimensional problems.]

The algorithm of the present paper utilizes a mathematically justified procedure for simulating from the joint (Θ×ℑ) posterior distribution (Eq. [18]). The basic idea is that, for a given hormone concentration profile, one generates an array of (e.g. 100) simulations from the posterior distribution, which allows one then to make probabilistic statements about the parameters of pulse times, secretion and elimination.

Summary of Algorithmic Flow

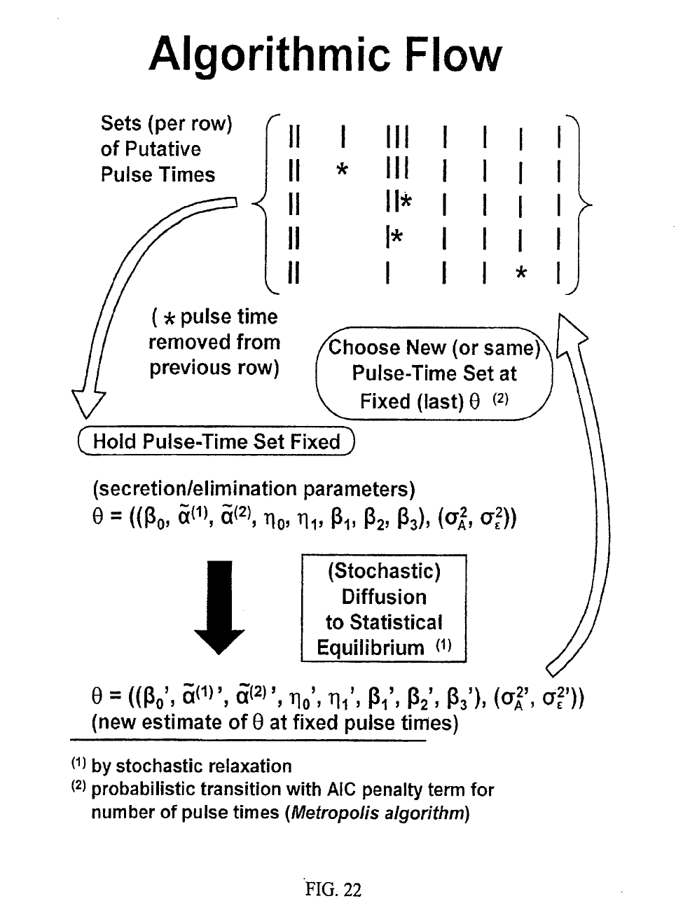

FIG. 22 schematizes the recursive algorithmic procedure, which technically proceeds as follows:

1. Selectively smooth hormone-concentration time series to produce a family of decremental potential pulse-time sets: ℑ={ℑ_{N}, ℑ_{N-1}, . . . , ℑₚ};

2. Randomly chose an initial pulse-time set from this family: ℑₘ;

3. Implement the iterative gradient-search equation [19]:

dθₜ=−∇h_{ℑ}_{ₘ}(θₜ|Y)dt+\sqrt{2}dBₜ, until “t is large”;

4. Move from ℑₘ, to a new set of pulse-onset times, ℑₘ₋₁, ℑₘ, or ℑₘ₊₁ via the Metropolis algorithm—ξ a constant, 0<ξ≦½, and θₜ the result of step 3:

P_{θ}(Tₘ₋₁|Tₘ)∝ξ min {1, exp[−(h_{ℑ}_{_{m−1}}(θₜ|Y)−h_{ℑ}_{ₘ}(θₜ|Y))]}

P_{θ}(Tₘ₊₁|Tₘ)∝ξ min {1, exp[−(h_{ℑ}_{ₘ₊₁}(θₜ|Y)−h_{ℑ}_{ₘ}(θₜ|Y))]}

P_{θ}(Tₘ|Tₘ)∝1−(sum of the above two)

5. Repeat Step 3 with the new pulse-time set and then step 4, recursively.

The validity of the algorithm has been established mathematically under the foregoing model conditions (Chattopadhyay, 2001). As implemented, the noise in Eq. [19] is fixed at a level that, asymptotic in interative time t, results in sampling from the posterior distribution.

Example Calculation: Posterior Distribution for Total Pulsatile Secretion (Eq. [9]).

Suppose that (\hat{θ}, \hat{ℑ}ₘ) is a “representative” simulation from the posterior distribution, Eq. [18]. Calculation of the conditional expectation E_{\hat{θ}} [A^{j}, j=1, . . . , m|Yᵢ, i=1, . . . , n] evaluated at \hat{θ} provides the basis for obtaining an estimate of the secretion rate Zᵢ=Z(tᵢ), i=1, . . . , n: (Eq. [12])

\hat{Z}ᵢ(i=1, . . . , n)=E_{\hat{θ}} [Zᵢ, i=1, . . . , n|Yᵢ, i=1, . . . , n].

One then obtains total secretion (basal and pulsatile) as:

$${\sum }_{i = 1}^{n} {\hat{Z}}_{i} , \qquad$$

total basal secretion as: n×\hat{β}₀, and total pulsatile as the difference. Repeated sampling of (\hat{θ},\hat{ℑ}ₘ), and calculation of total pulsatile secretion (for each), produces a probability histogram approximating the posterior distribution for total pulsatile secretion, given an observed concentration profile. Table 2 presents the posterior distributions for slow half-life, total basal, total pulsatile, number of pulses/day, and mass per pulse for the four concentration profiles of FIG. 1. Specifically, in reference to Eq. [9] (Methods), one has in the case of LH data in a young male:

P(80<24-hr Pulsatile LH Secretion (IU/L/day)<94|LH (young male data)=0.95 [20]

A convolution of estimated secretion rates with their sampled posterior values for biexponential kinetics yields reconstructed concentrations: Ŷᵢ, i=1, . . . , n.

TABLE 2
Quantiles (0.05, 0.25, 0.50, 0.75 and 0.95) and [Mean, SD] of Secretary
and Kinetic Parameters Calculated from their Posterior Distributions for Four Pituitary
Hormones.

| | | | Daily Pulsatile |
|---|---|---|---|
| | Slow Half-Life | Daily Basal Secretion | Secretion |
| ACTH | (9, 11, 15, 19, 25) | (0, 26, 134, 222, 327) | (595, 715, 797, 946, 1032) |
| | [15, 5] | [144, 115] | [825, 144] |
| LH | (114, 115, 116, 120, 125) | (0, 0, 0, 0, 0) | (80, 87, 93, 94, 94) |
| (Young male) | [117, 8] | [1, 7]* | [90, 5] |
| LH | (19, 21, 26, 86, 135) | (0, 67, 575, 617, 681) | (282, 364, 525, 690, 752) |
| (Older female) | [48, 39] | [422, 264] | [519, 180] |
| GH | (28, 31, 31, 31, 32) | (0, 0, 0, 0, 0) | (71, 72, 73, 73, 77) |
| | [30, 2] | [0, 0] | [73, 2] |
| | # Pulses/24 Hr | Mass Per Pulse | Changepoint |
| ACTH | (15, 22, 22, 25, 26) | (27, 30, 42, 51, 74) | (409, 890, 1021, 1333, 1429) |
| | [23, 3] | [44, 14] | [1001, 304] |
| LH | (12, 12, 12, 12, 14) | (6, 7, 8, 8, 8) | |
| (Young male) | [12, 2] | [7, 0.7] | |
| LH | (22, 25, 27, 27, 30) | (11, 19, 44, 50, 58) | |
| (Older female) | [26, 3] | [36, 16] | |
| GH | (17, 17, 17, 17, 20) | (3, 4, 4, 4, 4) | |
| | [17, 1] | [4, 0, 4] | |

*(2 non-zero values influenced this estimate)

Results

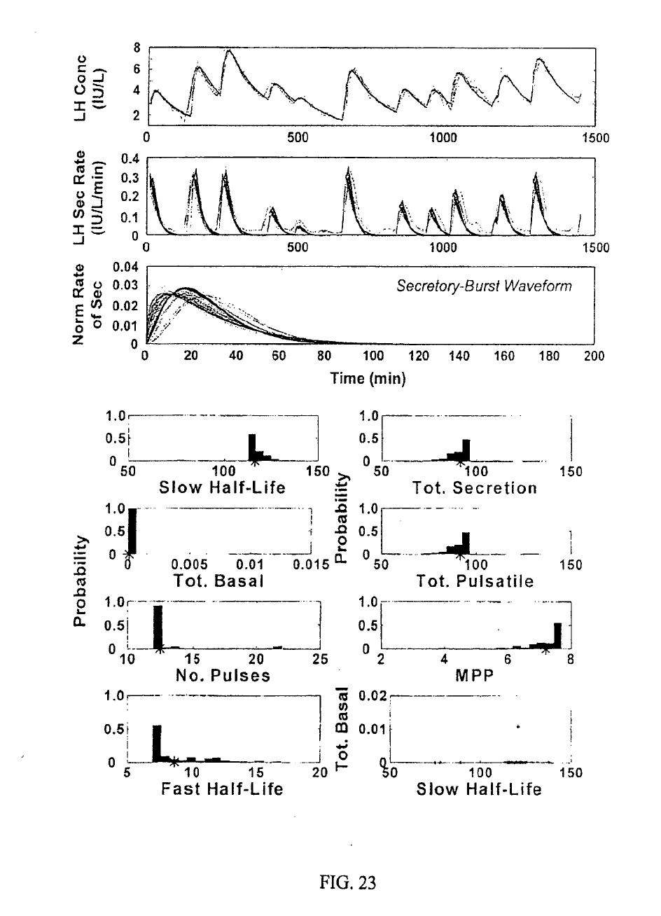

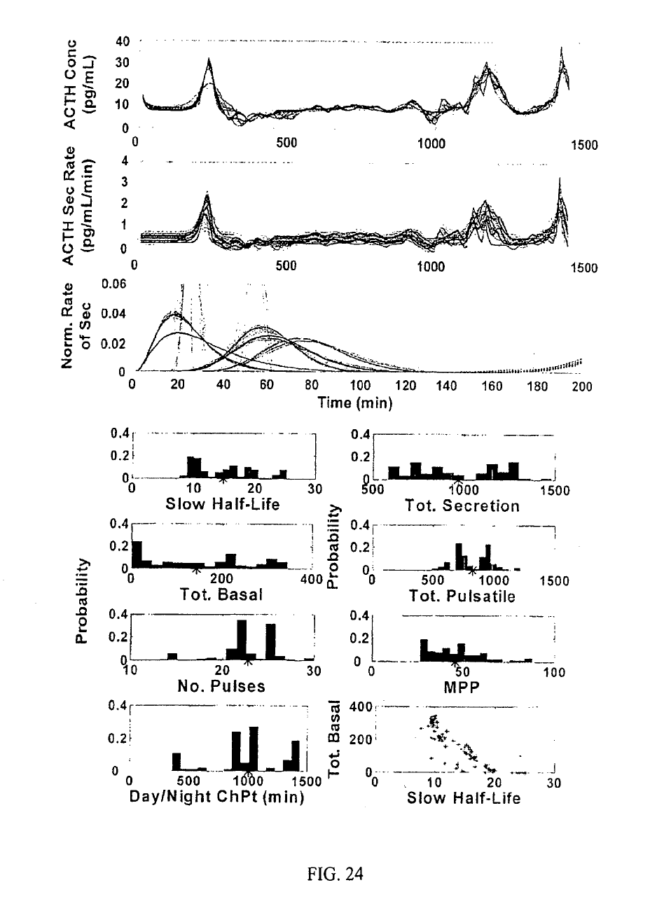

To illustrate algorithmic application, FIGS. 23 and 24 (top) present reconstructions of individual hormone time series from FIG. 1; viz., LH in a young man and ACTH in a woman. Individual panels for a given 24-hr profile include 100 estimates of each of: (i) the reconvolved concentration profile; (ii) the time course of calculated secretion; and (iii) the secretory-burst waveform (psi function). The several data series illustrate a spectrum of relative partitioning of total secretion into pulsatile and basal components; secretory-burst number, timing, mass and shape; elimination kinetics; and random variability, thus confirming algorithmic generality.

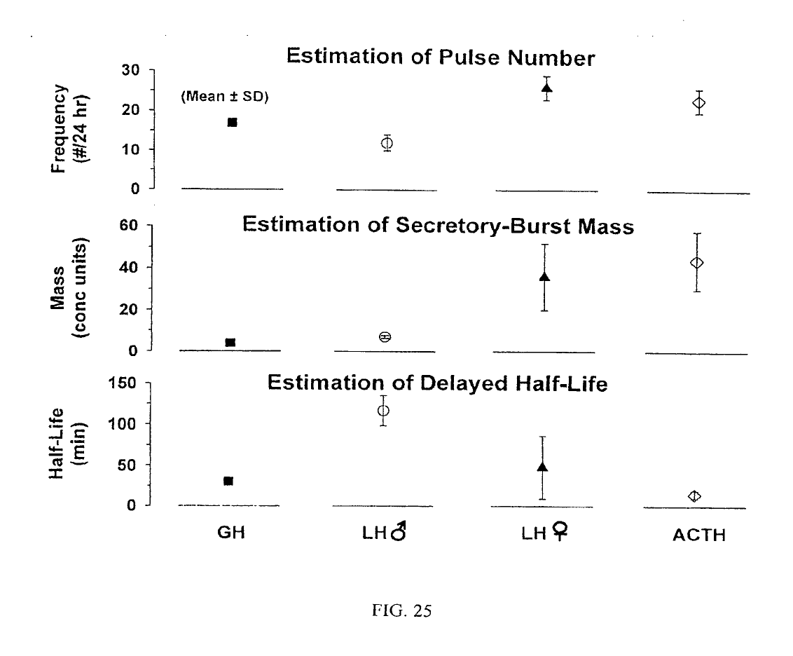

FIGS. 23 and 24 (bottom) give probability estimates of key parameters of the pituitary-hormone time series in FIGS. 4-5 (top). Histograms are based on 100 simulations from the joint posterior distribution. Several features are notable by inspection. First, waveform shape (3-parameter Gamma function) and pulse number (events/24 hr) are well determined probabilistically. Second, half-lives differ by hormone type from 15 min (ACTH) to 118 min (LH male) in these illustrative data sets (Table 1). Third, higher fractional basal secretion introduces greater variability in probability estimates of the slow half-life due to stronger parameter correlations, as inferred from the ACTH and postmenopausal LH profiles. And, fourth, under a model extension that allows two ACTH secretory-burst waveforms to evolve in separate time intervals contained in 24 hr (Keenan & Veldhuis, 2003), the estimated changepoint time of burst-shape is probabilistically consistent within an individual. Probability statements defined by quantities are made in Table 2 for the foregoing four profiles. The visual representation of the mean (of 100) estimate(s) and SD for each of the four individual time series is given in FIG. 25.

Biological signals are often intermittent (pulsatile), rather than continuous. In neurohormonal systems, both the frequency and amplitude of discrete pulses can convey significantly regulatory information to target tissues. For example, hypothalamic peptidyl signals are released episodically to the anterior pituitary gland, allowing recurrent stimulatory or inhibitory cycles without desensitization. Pituitary hormones, such as luteinizing hormone (LH), growth hormone (GH) and adrenocorticotropin hormone (ACTH) are secreted in bursts, which permit repeated cellular activation and recovery of second-messenger signaling pathways (Farhy & Veldhuis, 2003; Keenan et al., 2001; Keenan et al., 2004). Indeed, strong gender differences in nuclear transcriptional activity and specific promoter utilization in part reflect the pulsatile vis a vis continuous input of gonadotropin-releasing hormone to gonadotropes and GH to liver, muscle and fat (Urban et al., 1988; Giustina & Veldhuis, 1998). For these reasons, a host of experimental studies requires valid estimation of the number and amplitude of distinguishable neuroendocrine pulses and the relative contribution of basal (continuous) neurohormone release to the total signal. To this end, we have developed a model that combines discrete (pulse number), continuous (secretion and elimination rates) and stochastic (measurement and low-order biological variability) contributions by way of composite (simultaneous) signal reconstruction.

Quantifying intermittent neurohormone signaling provides a window into the physiological basis of pulse generation and feedforward and feedback control. Here, we assume that neuroglandular secretion comprises an unknown admixture of basal (time-invariant) and pulsatile (burst-like) release. The basal component putatively arises via constitutive neuropeptide release (Aryan et al., 1991), whereas the pulsatile component reflects secretory bursts that are timed by an apparently stochastic sequence of pulse times (e.g., a renewal-like process). Diffusion, distribution and elimination dissipate the secreted hormone in blood, tissue fluids and metabolic organs. And, sampling and measurement errors and biological nonuniformities introduce random variations into experimental observations. For these reasons, the present analytical strategy is jointly model-based (structural) and criterion-defined (statistical). The outcome is a conjoint estimate of basal and burst-like neurohormone secretion, elimination kinetics, pulse number and timing. Distinctive methodological aspects include: (a) a biologically motivated model form; (b) statistical estimation of all parameters jointly; (c) judicious assignment of random effects; and (d) probabilistic (Bayesian) reconstruction of the posterior distribution of each parameter for any given hormone profile.

Illustrative analyses of four intensively sampled neuroendocrine time series show a rich diversity of secretory-burst number, mass and shape; relative partitioning of basal and pulsatile secretion; biexponential elimination kinetics; and variability of hormone release over 24 hr (e.g., LH in a young man [FIG. 23] and ACTH in a woman [FIG. 24]). The Bayesian framework allows probabilistic estimates of each regulated dynamic realized in any given time series [see Table 2 (and FIG. 25) and histograms, FIGS. 24 and 25]. Statistical sampling revealed relatively narrow probability distributions for pulse number and secretory-burst waveform. On the other hand, the strong cross-correlation between basal secretion and slow-elimination half-life tends to widen the probability intervals for these parameters, as in the case of ACTH and postmenopausal LH. One approach to address this issue under a Bayesian formulation would be to utilize population-based kinetic estimates as prior constraints on the statistical solution. Some hormones, like testosterone, GH, and cortisol bind reversibly to one or more plasma proteins (Evans et al. 1992; Urban et al. 1988; Giustina & Veldhuis, 1998). The present methodology incorporates such exchange processes, when rate constants are reasonably known in whole plasma at 37° C. Examples are given for testosterone and cortisol in (Keenan & Veldhuis, 2004; Keenan et al. 2004).

In summary, the time-varying nature of numerous (nonconstitutive) biological signals can pose an impasse in studies of interglandular and intercellular signaling control. Signal control can be a preeminent feature of mammalian homeostatic adjustments within cells, between cells and among tissues. Two questions can be addressed experimentally and clinically: (a) what are the putative underlying signal (pulse or burst) properties, which putatively reflect endogenous regulatory mechanisms; and (b) how do well defined investigative probes modify specific signal properties? Both queries can involve objective dissection of the observed (composite) signal into its underlying components. The latter notably includes elements that are discrete (pulse number), continuous (rates of secretion and elimination, shape of the burst-like release episode), and stochastic (apparently random perturbations in measurements and short-term system behavior). We illustrate an objective Bayesian platform combining these 3 biological features in a valid composite solution. Accordingly, the framework presented here can have broader utility in other biological systems characterized by intermittent signal exchange.

A number of embodiments have been described. Nevertheless, it will be understood that various modifications may be made without departing from the spirit and scope of the disclosure. For example, other multisignal systems, including but not limited to hormonal axes, metabolite and substrate coregulation via enzymatic interconversions, pulmonary-respiratory gas inflow and exchange, and cardiovascular reflexes are directly amenable to analysis by the same model platform. It is to be understood that while the inventive concepts have been described in conjunction with the detailed description thereof, the foregoing description is intended to illustrate and not limit the scope of the concepts, which are defined by the scope of the appended claims. Other aspects, advantages, and modifications are within the scope of the claims.

### REFERENCES

Alvarez, L., Lions, P. L. and Morel, J. M. 1992. Image selective smoothing and edge detection by nonlinear diffusion. II. SIAM J. Numer. Anal. 29, 845-866.
Arvan, P., Kuliawat, R., Prabakaran, D., Zavacki, A. M., Elahi, D., Wang, S., Pilkey, D., 1991. Protein discharge from immature secretory granules displays both regulated and constitutive characteristics. J. Biol. Chem. 266, 14171-14174.
Butler, J. P., Spratt, D. I., O'Dea, L. St. L., Crowley, Jr. W. F., 1986. Interpulse interval sequence of LH in normal men essentially constitutes a renewal process. Am. J. Physiol. 250, E338-E340.
Camproux, A. C., Thalabard, J. C., Thomas, G., 1994. Stochastic modeling of the hypothalamic pulse generator activity. Am. J. Physiol. 267, E795-E800.
Chattopadhyay, S., 2001. Simultaneous Hormone Pulse Time and Secretion/Elimination Estimation: An Alternating Metropolis and Diffusion Scheme. Ph.D thesis, Department of Statistics, University of Virginia, Charlottesville, Va.
Clarke, I., Moore, L., Veldhuis, J. D., 2002. Intensive direct cavernous sinus sampling identifies high-frequency nearly random patterns of FSH secretion in ovariectomized ewes: combined appraisal by radioimmunoassay and bioassay. Endocrinol. 143, 117-129.
Clifton, D. K., Steiner, R. A., 1983. Cycle detection: a technique for estimating the frequency and amplitude of episodic fluctuations in blood hormone and substrate concentration. Endocrinol. 112, 1057-1064.
Diggle, P. J., Zeger, S. L., 1989. A Non-Gaussian Model for Time Series with Pulses. J Am Stat Assoc 84, 354-359.
Evans, W. S., Sollenberger, M. J., Booth Jr., R. A., Rogol, A. D., Urban, R. J., Carlsen, E. C., Johnson, M. L., Veldhuis, J. D., 1992. Contemporary aspects of discrete peak detection algorithms. II. The paradigm of the luteinizing hormone pulse signal in women. Endocr. Rev. 13, 81-104.
Farhy, L. S., Veldhuis, J. D., 2003. Joint pituitary-hypothalamic and intrahypothalamic autofeedback construct of pulsatile growth hormone secretion. Am. J. Physiol. Regul. Integr. Comp. Physiol. 285, R1240-R1249.
Foresta, C., Bordon, P., Rossato, M., Mioni, R., Veldhuis, J. D., 1997. Specific linkages among luteinizing hormone, follicle stimulating hormone, and testosterone release in the peripheral blood and human spermatic vein: evidence for both positive (feed-forward) and negative (feedback) within-axis regulation. J. Clin. Endocrinol. Metab. 82, 3040-3046.
Giustina, A., Veldhuis, J. D., 1998. Pathophysiology of the neuroregulation of growth hormone secretion in experimental animals and the human. Endocr. Rev. 19, 717-797.
Goodman, R. I., Karsch, F. J., 1980. Pulsatile secretion of luteinizing hormone: differential suppression by ovarian steroids. Endocrinol. 107, 1286-1290.
John, F. Partial Differential Equations. 1982. Fourth Edition. Springer-Verlag. New York.
Keenan, D. M., Alexander, S. L., Irvine, C. H. G., Clarke, I. J., Canny, B. J., Scott, C. J., Tilbrook, A. J., Turner, A. I., Veldhuis, J. D., 2004. Reconstruction of in vivo time-evolving neuroendocrine dose-response properties unveils admixed deterministic and stochastic elements in interglandular signaling. Proc. Natl. Acad. Sci. USA. 101, 6740-6745.
Keenan, D. M., Evans, W. S., Veldhuis, J. D., 2003. Control of LH secretory-burst frequency and interpulse-interval regularity in women. Am. J. Physiol. 285, E938-948.
Keenan, D. M., Licinio, J., Veldhuis, J. D., 2001. A feedback-controlled ensemble model of the stress-responsive hypothalamo-pituitary-adrenal axis. Proc. Natl. Acad. Sci. USA. 98, 4028-4033.
Keenan D. M., Roelfsema F., Veldhuis J. D., 2004. Endogenous ACTH concentration-dependent drive of pulsatile cortisol secretion in the human. Am. J. Physiol. 287, E652-E661.
Keenan, D. M., Sun, W., Veldhuis, J. D., 2000. A stochastic biomathematical model of the male reproductive hormone system. SIAM. J. Appl. Math. 61, 934-965.
Keenan, D. M., Veldhuis, J. D., 1997. Stochastic model of admixed basal and pulsatile hormone secretion as modulated by a deterministic oscillator. Am. J. Physiol. 273, R1182-R1192.
Keenan, D. M., Veldhuis, J. D., 1998. A biomathematical model of time-delayed feedback in the human male hypothalamic-pituitary-Leydig cell axis. Am. J. Physiol. 275, E157-E176.
Keenan, D. and Veldhuis, J. D. 2001. Disruptions in the hypothalamic luteinizing-hormone pulsing mechanism in aging men. Amer. J. Physiol., 281: R1917-R1924.
Keenan, D. M., Veldhuis, J. D., 2003. Cortisol feedback state governs adrenocorticotropin secretory-burst shape, frequency and mass in a dual-waveform construct: time-of-day dependent regulation. Am. J. Physiol. 285, R950-961.
Keenan, D. M., Veldhuis, J. D., 2004. Divergent gonadotropin-gonadal dose-responsive coupling in healthy young and aging men. Am. J. Physiol. 286, R381-R389.
Keenan, D. M., Veldhuis, J. D., Yang, R., 1998. Joint recovery of pulsatile and basal hormone secretion by stochastic nonlinear random-effects analysis. Am. J. Physiol. 275, R1939-R1949.
Kushler, R. H., Brown, M. B., 1991. A model for the identification of hormone pulses. Stat. Med. 10, 329-340.
Mauger, D. T., Brown, M. B., Kushler, R. H., 1995. A comparison of methods that characterize pulses in a time series. Stat. Med. 14, 311-325.
Merriam, G. R., Wachter, K., 1982. Algorithms for the study of episodic hormone secretion. Am. J. Physiol. 243, E310-E318.
Morel, J., Solimini, S., 1995. Variational methods in image segmentation. Birkhauser, Boston.
Munson, P. J., Rodbard, D., 1989. Pulse detection in hormone data: simplified, efficient algorithm. In: Proceedings of the Statistical Computing Secretion of the American Statistical Association. Washington, D.C., pp. 295-300.
O'Sullivan, F., O'Sullivan, J., 1988. Deconvolution of episodic hormone data: an analysis of the role of season on the onset of puberty in cows. Biometrics. 44, 339-353.
Oerter, K. E., Guardabasso, V., Rodbard, D., 1986. Detection and characterization of peaks and estimation of instantaneous secretory rate for episodic pulsatile hormone secretion. Comp. Biomed. Res. 19, 170-191.
Pincus, S. M., Mulligan, T., Iranmanesh, A., Gheorghiu, S., Godschalk, M., Veldhuis, J. D., 1996. Older males secrete luteinizing hormone and testosterone more irregularly, and jointly more asynchronously, than younger males. Proc. Natl. Acad. Sci. USA. 93, 14100-14105.
Redekopp, C., Irvine, C. H., Donald, R. A., Livesey, J. H., Sadler, W., Nicholls, M. G., Alexander, S. L., Evans, M. J., 1986. Spontaneous and stimulated adrenocorticotropin and vasopressin pulsatile secretion in the pituitary venous effluent of the horse. Endocrinol. 118, 1410-1416.
Santen, R. J., Bardin, D. W., 1973. Episodic luteinizing hormone secretion in man: pulse analysis, clinical interpretation and pathological mechanisms. J. Clin. Invest. 72, 2031.
Urban, R. J., Evans, W. S., Rogol, A. D., Kaiser, D. L., Johnson, M. L., Veldhuis, J. D., 1988. Contemporary aspects of discrete peak detection algorithms. I. The paradigm of the luteinizing hormone pulse signal in men. Endocr. Rev. 9, 3-37.
Urquhart, J., Li, C. C., 1968. The dynamics of adrenocortical secretion. Am. J. Physiol. 214, 73-85.
Van Cauter, E., 1981. Quantitative methods for the analysis of circadian and episodic hormone fluctuations. In: Van Cauter, E., Copinschi, G. (Eds.), Human Pituitary Hormones: Circadian and Episodic Variations. Nyhoff, The Hague, pp. 1.
Veldhuis, J. D., Carlson, M. L., Johnson, M. L., 1987. The pituitary gland secretes in bursts: appraising the nature of glandular secretory impulses by simultaneous multiple-parameter deconvolution of plasma hormone concentrations. Proc. Natl. Acad. Sci. USA. 84, 7686-7690.
Veldhuis, J. D., Fletcher, T. P., Gatford, K. L., Egan, A. R., Clarke, I. J., 2002. Hypophyseal-portal somatostatin (SIRF) and jugular venous growth hormone secretion in the conscious unrestrained ewe. Neuroendocrinol. 75, 83-91.
Veldhuis, J. D., Johnson, M. L., 1986. Cluster analysis: A simple, versatile and robust algorithm for endocrine pulse detection. Am. J. Physiol. 250, E486-E493.
Veldhuis, J. D., Johnson, M. L., 1992. Deconvolution analysis of hormone data. Meth Enzymol. 210, 539-575.
Veldhuis, J. D., Johnson, M. L., 1995. Specific methodological approaches to selected contemporary issues in deconvolution analysis of pulsatile neuroendocrine data. Meth. Neurosci. 28, 25-92.

What is claimed is:

1. A method for assessing an inter-dependent biological system within a mammal, comprising:
determining whether or not a set comprising at least three variables is within a diagnostic coordinate range;
wherein said set is obtained, using a mathematical formalism, from a measured value of a first constituent in a sample from said mammal and from a measured value of a second constituent in a sample from said mammal;
wherein said first and said second constituents are constituents of said inter-dependent biological system, wherein said inter-dependent biological system comprises at least three constituents;
wherein said diagnostic coordinate range represents said inter-dependent biological system in normal function, wherein the presence of said set within said diagnostic coordinate range indicates that said inter-dependent biological system of said mammal is normal; and
wherein the absence of said set within said diagnostic coordinate range indicates that said inter-dependent biological system of said mammal is abnormal.

2. The method of claim 1, wherein the inter-dependent biological system is a tripartite system.

3. The method of claim 1, wherein the inter-dependent biological system comprises glucose, glucagon, and insulin.

4. The method of claim 1, wherein the first and second constituents are selected from the group consisting of glucose, glucagon, and insulin.

5. The method of claim 1, wherein the inter-dependent biological system comprises testosterone, gonadotropin, and luteinizing hormone.

6. The method of claim 1, wherein the first and second constituents are selected from the group consisting of testosterone, gonadotropin, and luteinizing hormone.

7. The method of claim 1, wherein the mammal is a human.

8. The method of claim 1, wherein said at least three variables of said set are hormone concentrations.

9. The method of claim 1, wherein the diagnostic coordinate range comprises control sets comprising said at least three variables, wherein said sets are obtained, using said mathematical formalism, from a measured value of said first constituent from a collection of samples from healthy control mammals, and from a measured value of said second constituent in a collection of samples from healthy control mammals.

10. The method of claim 9, wherein the range of variable values comprises variable values sampled from a population of mammals, wherein the inter-dependent biological system is within the condition of homeostasis.

11. The method of claim 1, wherein the mathematical formalism comprises:
(a) a time delay signal wherein one of said at least three constituents accumulate in a target tissue before initiating a biological response;
(b) a nonlinear equation that represents concentration-dependent processes, and saturable cellular uptake and signaling processes;
(c) a stochastic term, to represent known measurement uncertainty and biological variability in dose-response properties; and
(d) signal parameter estimation procedures.

12. The method of claim 11, wherein the signal parameter estimation procedure comprises maximum-likelihood and Bayesian approaches.

13. The method of claim 11, wherein the time delay signal is mathematically represented as a pulse, wherein the pulse is an administered dose of one of said at least three constituents.

14. The method of claim 13, wherein the administered dose comprises a dose of glucose.

15. The method of claim 13, wherein the administered dose comprises intravenously-administered glucose.

16. The method of claim 1, wherein said measured value of said first constituent is measured from said sample as an activity or concentration level.

17. A method for detecting the presence or absence of physiological regulatory failure in a mammal, comprising:
obtaining a biological sample, wherein the biological sample contains at least two constituents of an inter-dependent, tripartite biological system;
applying measured values of the two constituents to a mathematical formalism to obtain a set of deconvolved, time-independent variables within the biological system; and
comparing the set of deconvolved time-independent variables to a diagnostic space coordinate exemplifying the tripartite biological system in normal function, wherein the coordinate position of the set of deconvolved, time-independent variables upon the diagnostic space coordinate indicates the presence or absence of physiological regulatory failure in a mammal.

18. A computer program product tangibly embodied in an information carrier, the computer program product including instructions that, when executed, perform operations for assessing an inter-dependent biological system within a mammal, comprising:
determining whether or not a set comprising at least three variables is within a diagnostic coordinate range,
wherein said set is obtained, using a mathematical formalism, from a measured value of a first constituent in a sample from said mammal and from a measured value of a second constituent in a sample from said mammal,
wherein said first and said second constituents are constituents of said inter-dependent biological system, wherein said inter-dependent biological system comprises at least three constituents,
wherein said diagnostic coordinate range represents said inter-dependent biological system in normal function, wherein the presence of said set within said diagnostic coordinate range indicates that said inter-dependent biological system of said mammal is normal, and
wherein the absence of said set within said diagnostic coordinate range indicates that said inter-dependent biological system of said mammal is abnormal.

19. A computer program product tangibly embodied in an information carrier, the computer program product including instructions that, when executed, perform operations for detecting the presence or absence of physiological regulatory failure in a mammal, comprising:
obtaining a biological sample, wherein the biological sample contains at least two constituents of an inter-dependent, tripartite biological system;
applying measured values of two constituents of an obtained biological sample, wherein the biological sample contains at least two constituents of an inter-dependent, tripartite biological system, to a mathematical formalism to obtain a set of deconvolved, time-independent variables within the biological system; and
comparing the set of deconvolved time-independent variables to a diagnostic space coordinate exemplifying the tripartite biological system in normal function, wherein the coordinate position of the set of deconvolved, time-independent variables upon the diagnostic space coordinate indicates the presence or absence of physiological regulatory failure in a mammal.
